SEQUENCE LISTING (4 sequences)

SEQ ID NO: 1 - DNA, 22 bp, Artificial Sequence
  Immunomodulatory nucleic acid sequence
tgactgtgaacgttcgagatga

SEQ ID NO: 2 - DNA, 22 bp, Artificial Sequence
  Control nucleic acid sequence
tgactgtgaaggttcgagatga

SEQ ID NO: 3 - DNA, 1137 bp, Artificial Sequence
  hssHADelta36Amba1 nucleic acid sequence
atgaaggctaagctgctggtcctcctttgcgccctgagcgctaccgacgcagatgagacaaggcgcttgactacctccggagcttacaacattattgatggctgttggagggggaaggcagattgggccgaaaaccgcaaggcactggctgactgtgcccaaggttttggcaaaggtaccgtcggtggcaaggacggagacatttacaccgtcacctccgagctcgacgacgatgttgctaaccccaaggagggcacactcagatttggggccgcccagaacaggcccctgtggatcattttcgagcgcgacatggtcatccgcctggacaaggagatggtcgtgaactctgacaaaactattgacggcagaggggctaaagttgagattattaatgcaggattcacattgaacggcgtgaagaatgtgatcatccacaatattaacatgcacgacgtcaaagtgaatcccggcgggttgatcaagagcaacgacggacccgctgcccccagagctggctctgacggtgacgctatctctatttctggcagcagccagatctggatcgaccactgcagcctgtctaaatctgtggatggcctggtggacgccaaattgggaaccactaggctgaccgtcagcaattctctcttcacacagcaccaattcgtcctgttgttcggggcaggggatgagaatatcgaagatagaggtatgctggctacagtggccttcaacaccttcaccgataatgtggaccagaggatgccaaggtgtcgccacgggttcttccaggtggtgaataacaactacgataagtggggctcctacgctattggtggctccgctagtccaactatcctcagccagggcaatcgcttctgtgcacccgacgagaggtctaagaagaacgttctggggagacacggtgaggccgccgccgagtccatgaagtggaactggcgcacaaacaaggacgtgcttgaaaatggcgcaattttcgttgccagcggggtggacccagttctcaccccagaacaaagcgctggtatgatccccgccgagcctggggagtccgccctgagcctcacatcttccgctggtgttctgtcctgccagcctggcgctccctgttga

SEQ ID NO: 4 - DNA, 22 bp, Artificial Sequence
  Control nucleic acid sequence
tgactgtgaaggttagagatga

(19) United States
(12) Patent Application Publication
(10) Pub. No.: US 2002/0142978 A1
(43) Pub. Date: Oct. 3, 2002

(54) Synergistic improvements to polynucleotide vaccines

(76) Inventors: Eyal Raz, Del Mar, CA (US); Kenji Takabayashi, San Diego, CA (US); Minh-Duc Nguyen, Oceanside, CA (US)

Correspondence Address: Carol L. Francis, BOZICEVIC, FIELD & FRANCIS LLP, Suite 200, 200 Middlefield Road, Menlo Park, CA, 94025, US

(21) Appl. No.: 09/828,505

(22) Filed: Apr. 6, 2001

Related U.S. Application Data
(60) Provisional application No. 60/195,890, filed on Apr. 7, 2000.

(51) Int. Cl. A61K 48/00

(52) U.S. Cl. 514/44

(57) ABSTRACT

The invention features a polynucleotide vaccine modified to enhance expression of the encoded antigen in host cells. The polynucleotide vaccine comprises an antigen-encoding nucleic acid sequence derived from a non-host species of a first phylum or first kingdom, wherein the native signal sequence of the antigen coding sequence is deleted and, optionally, replaced with a signal sequence of a polypeptide of a second phylum or a second kingdom that is functional in the host to be immunized (e.g., a viral signal sequence with a plant antigen-encoding sequence). In one embodiment, the signal sequence is a hemagglutinin A (HA) signal sequence, and the antigen is an allergen (e.g., plant allergen) or from a pathogen (e.g., a bacterium, virus or parasite). The polynucleotide vaccine of the invention provides a synergistic effect with an immunostimulatory sequence (ISS) adjuvant to not only maintain, but to enhance, the immune response to the encoded antigen.

### CROSS-REFERENCE TO RELATED APPLICATIONS

This application claims the benefit of prior U.S. provisional application serial No. 60/195,890, filed Apr. 7, 2000, which application is incorporated herein by reference in its entirety.

### GOVERNMENT RIGHTS

This invention was made with Government support under Grant No. a140682, awarded by the National Institutes of Health. The government may have certain rights in this invention.

### TECHNICAL FIELD OF THE INVENTION

The invention relates to a polynucleotide vaccine comprising a nucleic acid sequence encoding an antigen.

### BACKGROUND OF THE INVENTION

Gene vaccines offer a powerful tool for induction of immune response against viral and bacterial antigens as well as against various protein allergens. To make a productive and immunogenic vaccination vector, the cDNA of an antigen of interest is subcloned between the promoter and the terminator of a plasmid. This approach, however, does not always deliver an optimal or sufficient immune response in vivo. There remains a need to develop improved gene vaccines that elicit increased and more effective humoral and cellular immune responses.

Immunostimulatory DNA sequences (ISS) delivered in conjunction with an antigen activate innate immunity and bias the adaptive immune response toward Th1 differentiation, thus shifting the immune response away from a Th2 response, which includes immune responses associated with allergy. ISS have been used as an adjuvant to amplify the immune response to a co-delivered antigen. See, for example, WO 98/16247, and U.S. Pat. Nos. 5,736,524 and 5,780,448. The use of ISS, particularly at a high dose (e.g., greater than 10 μg), as an adjuvant with gene vaccines, however, has resulted in reduced antigen expression and failure to elicit immunostimulatory effects (Weeratna et al., 1998, Antisense & Nucleic Acid Drug Development 8:351-356). Thus, ISS have been considered useful with DNA vaccines only if the ISS is positioned within the DNA vaccine itself, either endogenously or through subcloning (Krieg et al., 1998, Trends Microbiol. 6(1):23-7; Weeratna et al., supra).

There is a need in the field for polynucleotide vaccines that provides for higher levels of production of the encoded antigen and improved immunogenicity. This is particularly true where the antigen is from a species different from that of the host (e.g., where the host is a mammal, the antigen is of non-mammalian origin). The present invention addresses this need.

### SUMMARY OF THE INVENTION

The invention features a polynucleotide vaccine modified to enhance expression of the encoded antigen in host cells. The polynucleotide vaccine comprises an antigen-encoding nucleic acid sequence derived from a non-host species of a first phylum or first kingdom, wherein the native signal sequence of the antigen coding sequence is deleted and, optionally, replaced with a signal sequence of a polypeptide of a second phylum or a second kingdom that is functional in the host to be immunized (e.g., a viral signal sequence with a plant antigen-encoding sequence). In one embodiment, the signal sequence is a hemagglutinin A (HA) signal sequence, and the antigen is an allergen (e.g., plant allergen) or from a pathogen (e.g., a bacterium, virus or parasite). The polynucleotide vaccine of the invention provides a synergistic effect with an immunostimulatory sequence (ISS) adjuvant to not only maintain, but to enhance, the immune response to the encoded antigen.

In preferred embodiments of the polynucleotide vaccine, at least one codon of the nucleic acid sequence encoding the antigen is modified from a wild type sequence of the non-host species to an analogous codon of a host species. If the host species is human, for example, the polynucleotide vaccine comprises a humanized codon bias.

In some embodiments, the polynucleotide vaccine further comprises all or an immunogenic fragment of a “universal antigen”, an antigen that most of the population has been immunized against by active immunization or through natural exposure. Examples of universal antigens include, but are not limited to, tetanus toxin, polio, diptheria, pertussis, measles and flu antigens. The polynucleotide encoding the universal antigen or immunogenic fragment thereof is preferably included in or fused with the polynucleotide encoding the antigen derived from a non-host species of a first phylum or first kingdom, or, in a less preferred embodiment, it can be delivered separately.

The invention additionally provides a method for modulating an immune response to an antigen, and a method for eliciting an immune response to an antigen. The method comprises administering to a subject a polynucleotide vaccine of the invention. The method preferably further comprises administering to the subject an immunostimulatory nucleotide sequence (ISS). Administration of both the polynucleotide vaccine and the ISS achieves a synergistic improvement in efficacy of the method. In one embodiment, the antigen is an allergen, such as a grass pollen or ragweed, latex, cat dander, food (such as peanut), house dust mite or cockroach allergen.

The ISS can be administered concurrently and/or prior to administration of the polynucleotide vaccine. Delivering ISS prior to vaccine administration provides additional advantages of a pre-priming effect whereby the efficacy of a vaccine is enhanced by earlier (e.g., 3 days to 3 weeks prior) treatment with ISS. Pre-priming is of particular interest where the host is at least substantially naive (e.g., has not been exposed to the antigen of the vaccine at a level sufficient to elicit a substantial immune response). The vaccine and the ISS can be administered via local or systemic routes, including topical, enteral and parenteral routes. Examples of enteral routes include, but are not limited to, oral, gastric or rectal administration. Examples of parenteral routes include, but are not limited to, intradermal, intramuscular, subcutaneous or intravenous administration. Preferably the polynucleotide vaccine is administered by intradermal injection. The polynucleotide vaccine and/or the ISS can be encapsulated in liposomes, microsomes or other microencapsulating materials as is known in the art.

In preferred embodiments, the ISS comprises a non-coding oligonucleotide sequence that may include at least one unmethylated CpG motif. Examples of an ISS include, but are not limited to, sequences comprising 5′-rrcgyy-3′, such as AACGTT, AGCGTT, GACGTT, GGCGTT, AACGTC, and AGCGTC, 5′-rycgyy-3′ such as GTCGTT, 5′-rrcgyycg-3′, 5′-rycgyycg-3′ or 5′-(TCG)ₙ-3′. A preferred ISS comprises 5′-AACGTTAG-3′, and more preferred is an ISS comprising 5′-AACGTTCG-3′.

### BRIEF DESCRIPTION OF THE FIGURES

FIG. 1 is a schematic illustration of a polynucleotide vaccine construct of the invention in which a cDNA encoding the short ragweed allergen Amb a1 is modified from its full-length of 396 codons by deletion of the 36 amino acid plant signal sequence and substitution with a 14 amino acid signal sequence derived from an influenza virus.

FIG. 2 is a series of 4 tables showing a comparison of codon usage between plants and humans.

FIG. 3 is a western blot showing expression of Amb a1 in Cos-7 cells using progressively modified polynucleotides. Lane 1 shows purified AgE. Lane 2 shows the baseline expression with unmodified Amb a1 in a pNDKm vector. Lane 3 shows a 3-fold increase in expression over baseline using the vector of lane 2 modified to delete the plant signal sequence. Lane 4 shows also a 3-fold expression increase over baseline using the vector with the plant signal sequence deleted and a hemagglutinin A signal sequence added. Lane 5 shows a 10-fold increase in expression over baseline using the same vector as in lane 4, but with a humanized codon bias.

FIG. 4A is a line graph showing Amb a1-specific IgG2a levels, in 10³ U/ml, in mice 0, 2, 4 and 6 weeks after immunization with pNDKm (open squares), pNDKm containing Amb a1 (open diamonds), pNDKm containing Amb a1 modified to substitute a hemagglutinin A signal sequence for the native Amb a1 36 amino acid signal sequence (open circles), and the same modified construct as above with a human codon bias (closed triangles).

FIG. 4B is a bar graph showing levels of Amb a1-specific interferon gamma (IFNγ), in pg/ml, released in vitro by CD4+ T cells of mice 6 weeks after immunization with pNDKm (first bar from left), pNDKm containing Amb a1 (second bar from left), pNDKm containing Amb a1 modified to substitute a hemagglutinin A signal sequence for the native Amb a1 36 amino acid signal sequence (third bar from left), and the same modified construct as above with a human codon bias (fourth bar from left).

FIG. 5A is a line graph showing Amb a1-specific IgG2a levels, in 10³ U/ml, in mice 0, 2, 4 and 6 weeks after immunization with pNDKm containing Amb a1 modified to substitute a hemagglutinin A signal sequence for the native Amb a1 36 amino acid signal sequence with the viral sequences and modified to humanize its codon bias. In addition to 50 μg of the hssHAΔ36Amb a1/pNDKm construct, the mice were co-injected with ISS-ODN in the following amounts: 0 μg (open squares), 0.4 μg (open diamonds), 2 μg (open circles), 10 μg (open triangles), or 50 μg (closed squares).

FIG. 5B is a bar graph showing levels of Amb a1-specific interferon gamma (IFNγ), in pg/ml, released in vitro by CD4+ T cells of mice 6 weeks after immunization with 50 μg of the hssHAΔ36Amb a1/pNDKm construct, and co-injected with ISS-ODN in the following amounts: 0 μg (first bar from left), 0.4 μg (second bar from left), 2 μg (third bar from left), 10 μg (fourth bar from left), or 50 μg (fifth bar from left).

FIG. 6 shows transcripts of IL-6 (first column), IL-12 (second column), and G3PDH (third column) detected by RT-PCR in mRNA isolated from spleen of mice 2 hours after intravenous injection with 200 μg of ISS-ODN, in either double-stranded form, having a native phosphodiester backbone (dsPO), or single-stranded form, having a synthetic, sulfur-containing phosphorothioate backbone (ssPS). For each gel, lane 1 represents mice treated with phosphate buffered saline (PBS) in lieu of ISS-ODN, lane 2 represents mice treated with ISS-ODN, and lane 3 represents mice treated with a mutant version of the ODN that lacks immunostimulatory sequences.

FIG. 7A is a graph showing reduction of Amb a1-specific IgE in vivo following administration of pNDKm/hssHAΔ36Amb a1 with ISS.

FIG. 7B is a schematic showing the immunization schedule for Example 6.

### DETAILED DESCRIPTION OF THE INVENTION

The present invention is based on the discovery that polynucleotide vaccines can be modified to boost expression of the encoded antigen, and further, that such modified vaccines can work synergistically with immunostimulatory oligonucleotides (ISS-ODN) to dramatically improve vaccine efficacy in vivo.

Advantageous modifications to a polynucleotide vaccine include deletion of a signal sequence native to the encoded antigen, insertion of a functional signal sequence compatible with the host that is derived from another species or from another kingdom of organism than the antigen, and biasing the usage of codons in the polynucleotide in accordance with the host species to be treated with the vaccine. Further improvement in efficacy can be obtained with the use of polynucleotides and immunostimulatory sequences in single-stranded form. The vaccines and methods of the invention are particularly advantageous for protection against infectious pathogens, such as bacteria, viruses and parasites, and for allergic immunotherapy, such as ragweed and grass pollen allergies. In general, and without being held to theory, the polynucleotide vaccines of the invention provide for production of antigen so as to provide antigen in the extracellular environment and enhance the immunogenicity of the composition in a manner that is synergistic with the use of immunomodulatory nucleic acid in the composition.

Before the present invention is described in further detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited, and in order to describe more fully the state of the art to which this invention pertains.

It must be noted that as used herein and in the appended claims, the singular forms “a”, “and”, and “the” include plural referents unless the context clearly dictates otherwise. Thus, for example, reference to “an immunostimulatory nucleic acid molecule” includes a plurality of such molecules and reference to “the antigen” or “the allergen ” includes reference to one or more antigens or one or more allergens, and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The terms “oligonucleotide,” “polynucleotide,” and “nucleic acid molecule”, used interchangeably herein, refer to a polymeric forms of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Polynucleotides” also encompasses modified polynucleotides, including but not limited to, modifications of the 3′OH, 5′OH, or both the 3′ and 5′OH groups, modification f the nucleotide base, modifications of the sugar component, and modifications of the phosphate group. For example, the backbone of the polynucleotide can comprise sugars and phosphate groups (as may typically be found in RNA or DNA), or modified or substituted sugar or phosphate groups. Alternatively or in addition, the backbone of the polynucleotide can comprise a polymer of synthetic subunits such as phosphoramidites, and/or phosphorothioates, and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323.

A polynucleotide may comprise modified nucleotides, such as methylated nucleotides and nucleotide analogs, uracyl, other sugars, and linking groups such as fluororibose and thioate, and nucleotide branches. The polynucleotide may comprise one or more L-nucleosides. Polynucleotides can also comprise at least one nucleoside comprising an L-sugar. The L-sugar may be deoxyribose, ribose, pentose, deoxypentose, hexose, deoxyhexose, glucose, galactose, arabinose, xylose, or a sugar “analog” cyclopentyl group. The L-sugar may be in apyranosyl or furanosyl form. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

The terms “polypeptide,” “peptide,” and “protein”, used interchangeably herein, refer to a polymeric form of amino acids of any length, which can include coded and non-coded amino acids, chemically or biochemically modified or derivatized amino acids, and polypeptides having modified peptide backbones. The term includes polypeptide chains modified or derivatized in any manner, including, but not limited to, glycosylation, formylation, cyclization, acetylation, phosphorylation, and the like. The term includes naturally-occurring peptides, synthetic peptides, and peptides comprising one or more amino acid analogs. The term includes fusion proteins, including, but not limited to, fusion proteins with a heterologous amino acid sequence, fusions with heterologous and homologous signal sequences, with or without N-terminal methionine residues; immunologically tagged proteins; and the like.

The terms “antigen” and “epitope” are well understood in the art and refer to the portion of a macromolecule which is specifically recognized by a component of the immune system, e.g., an antibody or a T-cell antigen receptor. As used herein, the term “antigen” encompasses antigenic epitopes, e.g., fragments of an antigen which are antigenic epitopes. Epitopes are recognized by antibodies in solution, e.g. free from other molecules. Epitopes are recognized by T-cell antigen receptor when the epitope is associated with a class I or class II major histocompatibility complex molecule.

As used herein the term “isolated” is meant to describe a compound of interest (e.g., a virus, a peptide, etc.) that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, preferably 75% free, and most preferably 90% free from other components with which it is naturally associated.

The terms “immunomodulatory nucleic acid molecule,” “immunostimulatory nucleic acid molecule,” “immunostimulatory oligonucleotide sequence,” “immunostimulatory polynucleotide sequence,” “immunomodulatory polynucleotide sequence,” “ISS,” “ISS-PN,” and “ISS-ODN,” are used interchangeably herein to refer to a polynucleotide that comprises at least one immunomodulatory nucleic acid moiety. “ISS” is often used for ease of reference and clarity, but is not meant to be limiting. The terms “immunomodulatory,” and “immunostimulatory,” as used herein in reference to a nucleic acid molecule, refer to the ability of a nucleic acid molecule to modulate an immune response in a vertebrate host. In particular, these terms refer to the ability of an immunostimulatory nucleic acid molecule to increase an immune response in a vertebrate host, particularly to increase a CTL response, particularly an antigen-specific CTL response. Such nucleic acid molecules have at least one ISS moiety.

In general, an ISS moiety is a single-or double-stranded DNA or RNA oligonucleotide, usually having at least six nucleotide bases, and which may have, for example, a modified oligonucleotide, a sequence of modified nucleosides, or a modified phosphate backbone. Preferably, the ISS moieties comprise, or may be flanked by, a CG nucleotide sequence or a p(IC) nucleotide sequence, which may be palindromic. ISS is meant to encompass substantially purified ISS polynucleotides, either naturally occurring, synthetic, or recombination, as well as ISS-enriched nucleic acid, such as microbial DNA or plasmid DNA. Exemplary ISS moieties are described in more detail infra. Immunomodulatory nucleic acid encompasses substantially purified immunomodulatory nucleic acid as well as crude, detoxified bacterial (e.g., mycobacterial) RNA or DNA, as well as plasmids enriched for immunomodulatory nucleic acid molecules. In some embodiments, an “immunomodulatory sequence-enriched plasmid” refers to a linear or circular plasmid that comprises or is engineered to comprise a greater number of CpG motifs than normally found in mammalian DNA. Exemplary immunomodulatory sequence-enriched plasmids are described in, for example, Roman et al. (1997) Nat. Med. 3(8):849-54.

In general, immunomodulatory nucleic acid molecules do not provide for, nor is there any requirement that they provide for, expression of any amino acid sequence encoded by the immunomodulatory nucleic acid molecule. Thus the sequence of an immunomodulatory nucleic acid molecule may be, and generally is, non-coding. Immunomodulatory nucleic acid molecules may comprise a linear double or single-stranded molecule, a circular molecule, or can comprise both linear or circular segments. Immunomodulatory nucleic acid molecules may be single-stranded or double stranded, or may be completely or partially double-stranded.

In some embodiments, an immunomodulatory nucleic acid molecule of the invention is an oligonucleotide, e.g., has a sequence of from about 6 to about 200, from about 10 to about 100, from about 12 to about 50, or from about 15 to about 25 nucleotides in length. In other embodiments, the immunomodulatory nucleic acid molecule is part of a larger nucleotide construct (e.g., a plasmid vector, a viral vector, or other such construct). A wide variety of plasmid and viral vectors are known in the art, and need not be elaborated upon here. A large number of such vectors has been described in various publications, see, e.g., Current Protocols in Molecular Biology, (F. M. Ausubel et al., Eds. 1987, and updates). Many suitable plasmids and vectors are commercially available.

The terms, “increasing,” “inducing,” and “enhancing,” used interchangeably herein with reference to an aspect of an immune response (e.g., a Th1 response), refer to any increase in the recited aspect of the immune response over background (e.g., relative to untreated), and include inducing the immune response over an absence of a measurable parameter of the immune response, or increasing immune response over a previously measurable immune response.

The terms, “decreasing” and “inhibiting” are used interchangeably herein with reference to an aspect of an immune response (e.g., a Th2 response or allergic response), refer to any decrease in the recited aspect of the immune response over background (e.g., relative to untreated), and include, for example, in the context of allergy, decreasing IgE production, histamine release, or other measure of an allergic response following allergen challenge of an antigen-sensitive host, including decreasing the level of IgE, histamine release, or the like compared to a level of IgE, histamine, or the like in an untreated of an antigen-sensitive host.

As used herein, “subject” or “host” refers to the recipient of the vaccine or therapy to be practiced according to the invention. The subject can be any vertebrate, but will preferably be a mammal. If a mammal, the subject will preferably be a human, but may also be a domestic livestock, laboratory subject or pet animal. Exemplary subjects may include cattle, dogs, cats, guinea pigs, rabbits, rats, mice, horses, and the like.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

As used herein, “species” refers to species of organisms, unless clearly indicated otherwise. A non-host species is any species that differs from the species of the host. Likewise, “phylum” and “kingdom” refer to taxonomical classifications of organisms.

As used herein, “native”, in the context of nucleotide or amino acid sequence, refers to wild type or unaltered sequence, e.g., where the sequence is a coding sequence, a “native” sequence is a naturally-occurring sequence that encodes a functional gene product, e.g., a functional polypeptide.

As used herein, “analogous codon” means a codon that encodes the same amino acid, but may comprise a different triplet of bases.

As used herein, “universal antigen” refers to an antigen to which hosts are likely to have already developed an immune response. Hosts will have developed an immune response to such antigens either through active immunization (e.g., tetanus toxin, polio) or through natural exposure to a common pathogen (e.g., flu virus, bacteria).

An “allergy” generally refers to hypersensitivity of a subject to a substance (allergen). Allergic conditions include eczema, allergic rhinitis or coryza, hay fever, bronchial asthma, urticaria (hives) and food allergies, and other atopic conditions.

“Asthma” generally refers to a disorder of the respiratory system characterized by inflammation, narrowing of the airways and increased reactivity of the airways to inhaled agents. Asthma is frequently, although not exclusively associated with atopic or allergic symptoms.

As used herein, “ameliorating an allergic response” means a reduction, or attenuation of further increase, in allergic symptoms. Early phase symptoms include, but are not limited to, anaphylaxis, bronchospasm, itching, swelling, hyperemia, and mucoid discharge. Late stage symptoms are the result of cellular infiltration (PMN, lymphocytes, eosinophils) and inflammation, and include, but are not limited to, bronchospasm, itching, swelling, hyperemia, and mucoid discharge.

As used herein, “enhancing a Th1 immune response” in a subject may be evidenced by:

(1) a reduction in levels of IL-4, IL-5 or IL-13 measured before and after antigen challenge; or detection of lower (or even absent) levels of IL-4 in a treated subject as compared to an antigen-primed, or primed and challenged, control;

(2) an increase in levels of IL-12, IL-18 and/or IFN (α, β or γ) before and after antigen challenge; or detection of higher levels of IL-12, IL-18 and/or IFN (α, β or γ) in an ISS treated subject as compared to an antigen-primed or, primed and challenged, control;

(3) IgG2a antibody production in a treated subject (in mouse or equivalent thereof in another mammalian species);

(4) a reduction (or attenuation of further increase) in levels of antigen-specific IgE as measured before and after antigen challenge; or detection of lower (or even absent) levels of antigen-specific IgE in an ISS treated subject as compared to an antigen-primed, or primed and challenged, control; and/or

(5) induction of a cytotoxic T lymphocyte (“CTL”) response in a treated subject.

As used herein, “pharmaceutically acceptable carrier” includes any material which, when combined with an active ingredient of a composition, allows the ingredient to retain biological activity and without causing disruptive reactions with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, Chapter 43, 14th Ed., Mack Publishing Co., Easton Pa. 18042, USA).

### Immunomodulatory Nucleic Acid For Use With the Polynucleotide Vaccine

Immunomodulatory nucleic acid molecules are polynucleotides that modulate activity of immune cells, especially immune cell activity associated with a type-1 (Th1-mediated) or type-1 like immune response. Furthermore, immunomodulatory nucleic acid molecules of the present invention encompass polynucleotides that modulate an immune response to an antigen so as to provide for protection against subsequent exposure to the antigen, e.g., in the context of vaccination against a pathogen or in the context of allergic immunotherapy. The immunomodulatory nucleic acid (often referred to herein for convenience as ISS) useful in the invention includes an oligonucleotide, which can be a part of a larger nucleotide construct such as a plasmid.

The term “polynucleotide” therefore includes oligonucleotides, modified oligonucleotides and oligonucleosides, alone or as part of a larger construct. The polynucleotide can be single-stranded DNA (ssDNA), double-stranded DNA (dsDNA), single-stranded RNA (ssRNA) or double-stranded RNA (dsRNA). The ISS can include bacterial DNA, such as heat-killed Listeria or BCG, which provide ISS activity. The polynucleotide portion can be linearly or circularly configured, or the oligonucleotide portion can contain both linear and circular segments. Modifications of oligonucleotides include, but are not limited to, modifications of the 3′OH or 5′OH group, modifications of the nucleotide base, modifications of the sugar component, and modifications of the phosphate group.

Nucleic acid molecules comprising an immunomodulatory nucleic acid molecule which are suitable for use in the methods of the invention include an oligonucleotide, which can be a part of a larger nucleotide construct such as a plasmid. The term “polynucleotide” therefore includes oligonucleotides, modified oligonucleotides and oligonucleosides, alone or as part of a larger construct. The polynucleotide can be single-stranded DNA (ssDNA), double-stranded DNA (dsDNA), single-stranded RNA (ssRNA) or double-stranded RNA (dsRNA). The polynucleotide portion can be linearly or circularly configured, or the oligonucleotide portion can contain both linear and circular segments. Immunomodulatory nucleic acid molecules also encompasses crude, detoxified bacterial (e.g., mycobacterial) RNA or DNA, as well as ISS-enriched plasmids. “ISS-enriched plasmid” as used herein is meant to refer to a linear or circular plasmid that comprises or is engineered to comprise a greater number of CpG motifs than normally found in mammalian DNA. Exemplary ISS-enriched plasmids are described in, for example, Roman et al. (1997) Nat Med. 3(8):849-54. Modifications of oligonucleotides include, but are not limited to, modifications of the 3′OH or 5′OH group, modifications of the nucleotide base, modifications of the sugar component, and modifications of the phosphate group.

The immunomodulatory nucleic acid molecule can comprise ribonucleotides (containing ribose as the only or principal sugar component), deoxyribonucleotides (containing deoxyribose as the principal sugar component), or in accordance with the established state-of-the-art, modified sugars, L-sugars, or sugar analogs may be incorporated in the oligonucleotide of the present invention. Examples of a sugar moiety that can be used include, in addition to ribose and deoxyribose, pentose, deoxypentose, hexose, deoxyhexose, glucose, arabinose, xylose, lyxose, and a sugar “analog” cyclopentyl group. The sugar may be in pyranosyl or in a furanosyl form. In the modified oligonucleotides of the present invention, the sugar moiety is preferably the furanoside of ribose, deoxyribose, arabinose or 2′-O-methylribose, and the sugar may be attached to the respective heterocyclic bases either in I or J anomeric configuration.

The phosphorous derivative (or modified phosphate group) that can be attached to the sugar or sugar analog moiety in the modified oligonucleotides of the present invention can be a monophosphate, diphosphate, triphosphate, alkylphosphate, alkanephosphate, phosphoronthioate, phosphorodithioate or the like. The heterocyclic bases, or nucleic acid bases that are incorporated in the oligonucleotide base of the ISS can be the naturally occurring principal purine and pyrimidine bases, (namely uracil or thymine, cytosine, adenine and guanine, as mentioned above), as well as naturally occurring and synthetic modifications of said principal bases. Those skilled in the art will recognize that a large number of “synthetic” non-natural nucleosides comprising various heterocyclic bases and various sugar moieties (and sugar analogs) are available, and that the immunomodulatory nucleic acid molecule can include one or several heterocyclic bases other than the principal five base components of naturally occurring nucleic acids. Preferably, however, the heterocyclic base in the ISS is selected from uracil-5-yl, cytosin-5-yl, adenin-7-yl, adenin-8-yl, guanin-7-yl, guanin-8-yl, 4-aminopyrrolo [2,3-d] pyrimidin-5-yl, 2-amino-4-oxopyrolo [2,3-d] pyrimidin-5-yl, 2-amino-4-oxopyrrolo [2,3-d] pyrimidin-3-yl groups, where the purines are attached to the sugar moiety of the oligonucleotides via the 9-position, the pyrimidines via the 1-position, the pyrrolopyrimidines via the 7-position and the pyrazolopyrimidines via the 1-position.

Structurally, the root oligonucleotide of the immunomodulatory nucleic acid molecule is a non-coding sequence that can include at least one unmethylated CpG motif. The relative position of any CpG sequence in ISS with immunomodulatory activity in certain mammalian species is 5′-CG-3′ (i.e., the C is in the 5′ position with respect to the G in the 3′ position).

Immunomodulatory nucleic acid molecules generally do not provide for, nor is there any requirement that they provide for, expression of any amino acid sequence encoded by the polynucleotide, and thus the sequence of a immunomodulatory nucleic acid molecule may be, and generally is, non-coding. Immunomodulatory nucleic acid molecules may comprise a linear double or single-stranded molecule, a circular molecule, or can comprise both linear and circular segments. Immunomodulatory nucleic acid molecules may be single-stranded, or may be completely or partially double-stranded.

In some embodiments, an immunomodulatory nucleic acid molecule is an oligonucleotide, e.g., consists of a sequence of from about 6 to about 200, from about 10 to about 100, from about 12 to about 50, or from about 15 to about 25, nucleotides in length.

Exemplary consensus CpG motifs of immunomodulatory nucleic acid molecules useful in the invention include, but are not necessarily limited to:

5′-Purine-Purine-[C]-[G]-Pyrimidine-Pyrimidine-3′, in which the immunomodulatory nucleic acid molecule comprises a CpG motif flanked by at least two purine nucleotides (e.g., GG, GA, AG, AA, II, etc.,) and at least two pyrimidine nucleotides (CC, TT, CT, TC, UU, etc.);

5 ′-Purine-TCG-Pyrimidine-Pyrimidine-3′;

5′-[TCG]ₙ-3′, where n is any integer that is 1 or greater, e.g., to provide a poly-TCG immunomodulatory nucleic acid molecule (e.g., where n=3, the polynucleotide comprises the sequence 5′-TCGTCGTCG-3′); and

5′-Purine-Purine-CG-Pyrimidine-Pyrimidine-CG-3′.

5′-Purine-TCG-Pyrimidine-Pyrimidine-CG-3′

Exemplary DNA-based immunomodulatory nucleic acid molecules useful in the invention include, but are not necessarily limited to, polynucleotides comprising the following nucleotide sequences:

AACGCC, AACGCT, AACGTC, AACGTT;
GCGCC, AGCGCT, AGCGTC, AGCGTT;
ACGCC, GACGCT, GACGTC, GACGTT;
GCGCC, GGCGCT, GGCGTC, GGCGTT;
TCGCC, ATCGCT, ATCGTC, ATCGTT;
TCGCC, GTCGCT, GTCGTC, GTCGTT; and
CGTCG, and TCGTCGTCG.

Octameric sequences are generally the above-mentioned hexameric sequences, with an additional 3′CG. Exemplary DNA-based immunomodulatory nucleic acid molecules useful in the invention include, but are not necessarily limited to, polynucleotides comprising the following octameric nucleotide sequences:

ACGCCCG, AACGCTCG, AACGTCCG, AACGTTCG;
GCGCCCG, AGCGCTCG, AGCGTCCG, AGCGTTCG;
ACGCCCG, GACGCTCG, GACGTCCG, GACGTTCG;
GCGCCCG, GGCGCTCG, GGCGTCCG, GGCGTTCG;
TCGCCCG, ATCGCTCG, ATCGTCCG, ATCGTTCG;
TCGCCCG, GTCGCTCG, GTCGTCCG, and GTCGTTCG.

Immunomodulatory nucleic acid molecules useful in the invention can comprise one or more of any of the above CpG motifs. For example, immunomodulatory nucleic acid molecules useful in the invention can comprise a single instance or multiple instances (e.g., 2, 3, 5 or more) of the same CpG motif. Alternatively, the immunomodulatory nucleic acid molecules can comprises multiple CpG motifs (e.g. 2, 3, 5 or more) where at least two of the multiple CpG motifs have different consensus sequences, or where all CpG motifs in the immunomodulatory nucleic acid molecules have different consensus sequences.

A non-limiting example of an immunomodulatory nucleic acid molecule is one with the sequence 5′-TGACTGTGAACGTTCGAGATGA-3′ (SEQ ID NO:1). An example of a control nucleic acid molecule is one having the sequence 5′-TGACTGTGAAgGTTCGAGATGA-3′ (SEQ ID NO:2), which differs from SEQ ID NO:1 at the nucleotide indicated in lower case type.

Immunomodulatory nucleic acid molecules useful in the invention may or may not include palindromic regions. If present, a palindrome may extend only to a CpG motif, if present, in the core hexamer or octamer sequence, or may encompass more of the hexamer or octamer sequence as well as flanking nucleotide sequences.

The core hexamer structure of the foregoing immunomodulatory nucleic acid molecules can be flanked upstream and/or downstream by any number or composition of nucleotides or nucleosides. However, ISS are at least 6 bases in length, and preferably are between 6 and 200 bases in length, to enhance uptake of the immunomodulatory nucleic acid molecule into target tissues.

In particular, immunomodulatory nucleic acid molecules useful in the invention include those that have hexameric nucleotide sequences having “CpG” motifs. Although DNA sequences are generally preferred, RNA immunomodulatory nucleic acid molecules can be used, with inosine and/or uracil substitutions for nucleotides in the hexamer sequences.

### Modifications

Immunomodulatory nucleic acid molecules can be modified in a variety of ways. For example, the immunomodulatory nucleic acid molecules can comprise backbone phosphate group modifications (e.g., methylphosphonate, phosphorothioate, phosphoroamidate and phosphorodithioate internucleotide linkages), which modifications can, for example, enhance stability of the immunomodulatory nucleic acid molecule in vivo, making them particularly useful in therapeutic applications. A particularly useful phosphate group modification is the conversion to the phosphorothioate or phosphorodithioate forms of an immunomodulatory nucleic acid molecule. Phosphorothioates and phosphorodithioates are more resistant to degradation in vivo than their unmodified oligonucleotide counterparts, increasing the half-lives of the immunomodulatory nucleic acid molecules and making them more available to the subject being treated.

Other modified immunomodulatory nucleic acid molecules encompassed by the present invention include immunomodulatory nucleic acid molecules having modifications at the 5′ end, the 3′ end, or both the 5′ and 3′ ends. For example, the 5′ and/or 3′ end can be covalently or non-covalently conjugated to a molecule (either nucleic acid, non-nucleic acid, or both) to, for example, increase the bio-availability of the immunomodulatory nucleic acid molecules, increase the efficiency of uptake where desirable, facilitate delivery to cells of interest, and the like

### Preparation of Immunomodulatory Nucleic Acid Molecules

Immunomodulatory nucleic acid molecules can be synthesized using techniques and nucleic acid synthesis equipment well known in the art (see, e.g., Ausubel et al. Current Protocols in Molecular Biology, (Wiley Interscience, 1989); Maniatis et al. Molecular Cloning: A Laboratory Manual (Cold Spring Harbor Laboratories, New York, 1982); and U.S. Pat. Nos. 4,458,066; and 4,650,675. Individual polynucleotide fragments can be ligated with a ligase such as T4 DNA or RNA ligase as described in, e.g., U.S. Pat. No. 5,124,246. Oligonucleotide degradation can be accomplished through exposure to a nuclease, see, e.g., U.S. Pat. No. 4,650,675. As noted above, since the immunomodulatory nucleic acid molecules need not provide for expression of any encoded amino acid sequence, the invention does not require that the immunomodulatory nucleic acid molecules be operably linked to a promoter or otherwise provide for expression of a coding sequence.

Alternatively, immunomodulatory nucleic acid molecules can be isolated from microbial species (e.g., mycobacteria) using techniques well known in the art such as nucleic acid hybridization, amplification (e.g., by PCR), and the like. Isolated immunomodulatory nucleic acid molecules can be purified to a substantially pure state, e.g., free of endogenous contaminants, e.g., lipopolysaccharides. Immunomodulatory nucleic acid molecules isolated as part of a larger polynucleotide can be reduced to the desired length by techniques well known in the art, such as endonuclease digestion. Other techniques suitable for isolation, purification, and production of polynucleotides to obtain ISS will be readily apparent to the ordinarily skilled artisan in the relevant field.

Circular immunomodulatory nucleic acid molecules can also be synthesized through recombinant methods or chemically synthesized. Where circular immunomodulatory nucleic acid molecules are obtained through isolation or recombinant methods, the immunomodulatory nucleic acid molecule can be provided as a plasmid. Chemical synthesis of smaller circular oligonucleotides can be performed using methods known in the art (see, e.g., Gao et al. (1995) Nucl. Acids. Res. 23:2025-9; Wang et al., (1994) Nucl. Acids Res. 22:2326-33).

Where the immunomodulatory nucleic acid molecule comprises a modified oligonucleotide, the modified oligonucleotides can be synthesized using standard chemical techniques. For example, solid-support based construction of methylphosphonates has been described in Agrawal et al. Tet. Lett. 28:3539-42. Synthesis of other phosphorous-based modified oligonucleotides, such as phosphotriesters (see, e.g., Miller et al. (1971) J. Am Chem Soc. 93:6657-65), phosphoramidates (e.g., Jager et al. (1988) Biochem. 27:7237-46), and phosphorodithioates (e.g., U.S. Pat. No. 5,453,496) is known in the art. Other non-phosphorous-based modified oligonucleotides can also be used (e.g., Stirchak et al. (1989) Nucl. Acids. Res. 17:6129-41).

Preparation of base-modified nucleosides, and the synthesis of modified oligonucleotides using such base-modified nucleosides as precursors is well known in the art, see, e.g., U.S. Pat. Nos. 4,910,300; 4,948,882; and 5,093,232. These base-modified nucleosides have been designed so that they can be incorporated by chemical synthesis into either terminal or internal positions of an oligonucleotide. Nucleosides modified in their sugar moiety have also been described (see, e.g., U.S. Pat. Nos. 4,849,513; 5,015,733; 5,118,800; and 5,118,802).

Techniques for making phosphate group modifications to oligonucleotides are known in the art. Briefly, an intermediate phosphate triester for the target oligonucleotide product is prepared and oxidized to the naturally-occurring phosphate triester with aqueous iodine or other agents, such as anhydrous amines. The resulting oligonucleotide phosphoramidates can be treated with sulfur to yield phosphorothioates. The same general technique (without the sulfur treatment step) can be used to produced methylphosphoamidites from methylphosphonates. Techniques for phosphate group modification are well known and are described in, for example, U.S. Pat. Nos. 4,425,732; 4,458,066; 5,218,103; and 5,453,496.

### Identification of Immunomodulatory Nucleic Acid Molecules

Confirmation that a particular compound has the properties of an immunomodulatory nucleic acid molecule useful in the invention can be obtained by evaluating whether the immunomodulatory nucleic acid molecule elicits the appropriate cytokine secretion patterns, e.g., a cytokine secretion pattern associated with a type-1 immune response. ISS delivered with an antigen also induces activity of cytotoxic T cells and acts as a very strong mucosal adjuvant (see, e.g., Horner (1998) Cell. Immunol. 190:77-82). As noted above, immunomodulatory nucleic acid molecules of interest in the methods of the invention are those that elicit a Th1 -mediated response, and/or, where the antigen is an allergen, shift the immune response away from an allergic immune response.

In general, helper T (Th) cells are divided into broad groups based on their specific profiles of cytokine production: Th1, Th2, and Th0. “Th1” cells are T lymphocytes that release predominantly the cytokines IL-2 and IFN-γ, which cytokines in turn promote T cell proliferation, facilitate macrophage activation, and enhance the cytolytic activity of natural killer (NK) cells and antigen-specific cytotoxic T cells (CTL). In contrast, the cytokines predominantly released by Th2 cells are IL-4, IL-5, and IL-10. IL-4 and IL-5 are known to mediate antibody isotype switching towards IgE or IgA response, and promote eosinophil recruitment, skewing the immune system toward an “allergic” type of response. Th0 cells release a set of cytokines with characteristics of both Th1-type and Th2-type responses. While the categorization of T cells as Th1, TH2, or Th0 is helpful in describing the differences in immune response, it should be understood that it is more accurate to view the T cells and the responses they mediate as forming a continuum, with Th1 and Th2 cells at opposite ends of the scale, and Th0 cells providing the middle of the spectrum. Therefore, it should be understood that the use of these terms herein is only to describe the predominant nature of the immune response elicited, and is not meant to be limiting to an immune response that is only of the type indicated. Thus, for example, reference to a “type-1” or “Th1” immune response is not meant to exclude the presence of a “type-2” or “Th2” immune response, and vice versa.

Details of in vitro and in vivo techniques useful for evaluation of production of cytokines associated with a type-1 or type2 response, as well as for evaluation of antibody production, are well known in the art. Likewise, methods for evaluating the ability of candidate ISS to modulate an immune response are also well known in the art, and are further exemplified in the Examples below.

### Constructs For Use in the Invention

Constructs, as well as methods of making such constructs, suitable for delivery to a host and expression of the encoded antigen are well known in the art, see, e.g., U.S. Pat. Nos. 5,830,877; 5,804,566; 5,693,622; 5,679,647; 5,589,466; and 5,580,859.

In general, the antigen-encoding sequence can be provided in a plasmid vector or a viral vector, of which numerous suitable examples are known in the art and are commercially available. In one embodiment, the vector is a plasmid vector. Suitable plasmid vectors are well-known in the art and include the vectors described in Current Protocols in Molecular Biology, (F. M. Ausubel et al., Eds. 1987, and updates. Alternatively the construct can be based on a viral vector (e.g., a construct having sequences of viral origin).

In general, the construct minimally comprises: an antigen-encoding sequence;

and a promoter to facilitate expression in a cell of the host to which the construct is administered. The construct may further comprise an immunomodulatory sequence, or the immunomodulatory nucleic acid may be administered separately (e.g., either prior to or concomitant with administration of the vector). Exemplary antigens (including allergens) for use in the constructs of the invention are described in more detail below.

### Deletion of Native Sequences of Antigen-encoding Sequence to Enhance Expression in Host Cells

In one embodiment, the invention is based on the discovery that expression of the encoded antigen, and thus the immunogenic effect of administration of the encoding construct, is enhanced by deletion of a signal sequence native to the antigen. In general, “signal sequences” are short sequences that direct newly synthesized secretory or membrane proteins to and through membranes of the endoplasmic reticulum of the mammalian host cell. They are often, but not universally, in an N-terminal location and are cleaved off by signal peptidases after the protein has crossed the membrane. Signal sequences generally share three common structural features: 1) a hydrophobic core, known as the h-region, comprising at least eight uncharged residues flanked by 2) a polar basic region (n-region) on the N-terminal side, and 3) a hydrophilic region (c-region) of about six residues terminating at a small uncharged residue. This residue contributes the carboxy group of the peptide bond that is cleaved by signal peptidase. The signal sequence is also known as the leader peptide or translated leader sequence. Signal sequences are sometimes referred to in the art as translated leader sequences, and thus may sometimes be referred to as “leader sequences”. Methods for identifying signal sequences, based on sequence or based on function, are well known in the art. Likewise, methods for modification of a coding sequence to delete a desired sequence are also well-known in the art.

The invention also contemplates deletion of other sequences that may affect efficiency of transcription or translation in the host cell. For example, an increase in translational efficiency may also be obtained by deletion of untranslated leader sequences. Methods for identifying such untranslated sequences are well known in the art, as are methods of modification of nucleic acids to delete such untranslated leader sequences.

### Insertion of Heterologous Signal Sequence

In another embodiment, the invention is based on the discovery that expression of an encoded antigen, and thus the immunogenic effect of administration of the corresponding polynucleotide construct, is enhanced when the encoded antigen is operably linked to a polynucleotide encoding heterologous signal sequence. By “heterologous” is meant that the sequence encoding the signal sequence and the sequence encoding the antigen are from different species, usually different phyla or different kingdoms. This is particularly useful where the antigen is of a phylum or kingdom that is different from that of the host to which the vaccine is to be administered (e.g., where the host is mammalian and the antigen is a plant, insect, or other non-mammalian allergen).

For example, the antigen-encoding sequence can be from a non-host species of a first phylum or first kingdom, wherein the native signal sequence of the antigen coding sequence is replaced with a leader sequence derived from a polypeptide of a second phylum or a second kingdom (e.g., a viral leader sequence is used with a plant antigen). In one embodiment, the leader sequence is a hemagglutinin A (HA) leader sequence, and the antigen is from an allergen (e.g., plant allergen) or is from a pathogen, such as a bacterium, virus or parasite.

By “operably linked” in the context of an antigen-encoding sequence and a signal sequence is generally meant that the leader sequence-encoding polynucleotide is positioned relative to the antigen-encoding polynucleotide sequence so as to enhance production of the antigen. Although it may not be necessary to the invention, the resulting recombinant polypeptide may be post-translationally processed to separate at least a portion of the signal sequence polypeptide from the antigen polypeptide.

A signal sequence is usually encoded by nucleic acid encoding a secreted or membrane-bound polypeptide to direct the encoded polypeptide to the surface of the cell. The signal sequence usually comprises a stretch of hydrophobic residues. Such signal sequences can fold into helical structures. Membrane-bound polypeptides typically comprise at least one transmembrane region that possesses a stretch of hydrophobic amino acids that can transverse the membrane. Some transmembrane regions also exhibit a helical structure. Hydrophobic fragments within a polypeptide can be identified by using computer algorithms. Such algorithms include Hopp & Woods, Proc. Natl. Acad. Sci. USA (1981) 78:3824-3828; Kyte & Doolittle, J. Mol. Biol. (1982) 157: 105-132; and RAOAR algorithm, Degli Esposti et al., Eur. J. Biochem. (1990) 190: 207-219.

Of particular interest are polynucleotides encoding a signal sequence that is adapted for function in a mammalian cell. Exemplary signal sequences include, but are not necessarily limited to, mammalian signal sequences (e.g., signal sequences obtained from a mammalian nucleotide sequence or that are functional in mammalian cells, e.g., albumin), viral signal sequences (e.g., a signal sequence of influenza virus hemagglutinin A (HA) or other viral polypeptide), bacterial signal sequences that function in an mammalian host, and the like.

In addition, or alternatively, it may be desirable to operably link the antigen-encoding sequence to an untranslated leader sequence that functions in the host cell to enhance expression. Exemplary leader sequences that can be suitable for use in the invention include, but are not limited to, those described in U.S. Pat. No. 5,891,665, which describes untranslated leader sequences of RNA virus non-structural genes that act as enhancers of translation of mRNA.

### Modification of an Antigen-encoding Sequence to Provide for Human Codon Usage Bias

In another embodiment, the antigen-encoding sequence is modified so that the coding sequence more closely mimics the codon bias of the host to be treated. Of particular interest is the modification of the antigen coding sequence to reflect human codon bias. Methods for such modification are well known in the art, as are methods and tools for determining the codon usage bias of the host to be treated (e.g., for determining the human codon usage bias). In general, “codon usage bias” refers to the extent to which one codon is preferentially used to code for a particular amino acid over all other codons that code for that same amino acid.

Methods and tools (e.g., software tools) for analysis of the codon usage bias of a first organism compares to a second organism (e.g., humans versus plants) are well known in the art and publicly or commercially available. For example, A simple, effective measure of synonymous codon usage bias, the Codon Adaptation Index, is described by Sharp et al. (Nucleic Acids Res 1987 Feb 11;15(3):1281-95) detailed. The index uses a reference set of highly expressed genes from a species to assess the relative merits of each codon, and a score for a gene is calculated from the frequency of use of all codons in that gene. The index assesses the extent to which selection has been effective in molding the pattern of codon usage.

In another example, Wright (Gene 1990 Mar 1;87(1):23-9) describes a simple measure that quantifies how far the codon usage of a gene departs from equal usage of synonymous codons. This measure of synonymous codon usage bias, the “effective number of codons used in a gene”, Nc, can be easily calculated from codon usage data alone, and is independent of gene length and amino acid (aa) composition. Nc can take values from 20, in the case of extreme bias where one codon is exclusively used for each aa, to 61 when the use of alternative synonymous codons is equally likely. Nc thus provides an intuitively meaningful measure of the extent of codon preference in a gene. Codon usage patterns across genes can be investigated by the Nc-plot: a plot of Nc vs. G+C content at synonymous sites. Nc-plots are produced for Homo sapiens, Saccharomyces cerevisiae, Escherichia coli, Bacillus subtilis, Dictyostelium discoideum, and Drosophila melanogaster.

Additional tools for determining codon usage bias are found on the internet at, for example, a site described by James O. McInerney, which provides a program for evaluating codon usage in a set of genes (see also, McInerney, J. O., “GCUA: General Codon Usage Analysis,” (1998) “GCUA (General Codon Usage Analysis) Bioinformatics: 14 (4) 372-373). For additional methods of analysis, see, e.g., Shields et al. (1988) Mol. Biol. Evol. 5:704-716, describing scaled chi-squared analysis). See also, Kim et al. “Codon optimization for high-level expression of human erythropoietin (EPO) in mammalian cells” Gene (1997) 199(1-2):293-301; Makrides (1996) 60(3):512-538; Zolotukhin et al. (1996) J. Virol. 70(7):4646-54; Li et al. (1996) 181:11-124.

The table below compares the relative codon bias among plants (exemplified here by grass), humans, and E. coli. While the codon bias can vary according to the gene being examined, the table below provides some rough guidelines for relative codon usage in these organisms. In general, where the host is human, the antigen-encoding sequence is modified so as to modify codons that are infrequently used to a codon that is more frequently used in the human host. For example, in the table below, where the plant codon is TTT to encode phenylalanine, the codon is modified to be the codon TTC, the more frequently used codon in humans.

TABLE 2
Comparison of Codon Usage

| Amino Acid | Codon | Grasses (%) | Human (%) | E. coli (%) |
|---|---|---|---|---|
| Ala | GCT | 13 | 17 | 29 |
| | GCC | 48 | 53 | 15 |
| | GCA | 5 | 13 | 24 |
| | GCG | 33 | 17 | 32 |
| Arg | CGT | 3 | 7 | 6 |
| | CGC | 45 | 37 | 32 |
| | CGA | 3 | 6 | 1 |
| | CGG | 14 | 21 | 1 |
| | AGA | 10 | 10 | 0 |
| | AGG | 24 | 18 | 0 |
| Asn | AAT | 9 | 22 | 15 |
| | AAC | 91 | 78 | 86 |
| Asp | GAC | 12 | 25 | 45 |
| | GAG | 88 | 75 | 55 |
| Cys | TGT | 3 | 32 | 43 |
| | TGC | 97 | 68 | 57 |
| Gln | CAA | 8 | 12 | 16 |
| | CAG | 92 | 88 | 85 |
| Glu | GAA | 11 | 25 | 75 |
| | GAG | 89 | 75 | 25 |
| Gly | GGT | 8 | 12 | 54 |
| | GGC | 76 | 50 | 42 |
| | GGA | 6 | 14 | 2 |
| | GGG | 10 | 24 | 4 |
| His | CAT | 24 | 21 | 26 |
| | CAC | 76 | 79 | 74 |
| Ile | ATT | 6 | 44 | 30 |
| | ATC | 92 | 44 | 70 |
| | ATA | 2 | 11 | 0 |
| Leu | CTT | 9 | 5 | 4 |
| | CTC | 40 | 26 | 8 |
| | CTA | 0 | 3 | 0 |
| | CTG | 47 | 58 | 80 |
| | TTA | 0 | 2 | 3 |
| | TTG | 4 | 6 | 5 |
| Lys | AAA | 4 | 18 | 78 |
| | AAG | 96 | 82 | 22 |
| Met | ATG | 100 | 100 | 100 |
| Phe | TTT | 5 | 20 | 27 |
| | TTC | 95 | 80 | 74 |
| Pro | CCT | 10 | 19 | 10 |
| | CCC | 62 | 48 | 1 |
| | CCA | 10 | 16 | 13 |
| | CCG | 19 | 17 | 77 |
| Ser | TCT | 9 | 13 | 31 |
| | TCC | 50 | 28 | 29 |
| | TCA | 2 | 5 | 4 |
| | TCG | 20 | 9 | 8 |
| | AGT | 0 | 10 | 4 |
| | AGC | 19 | 34 | 25 |
| Thr | ACT | 9 | 14 | 28 |
| | ACC | 71 | 57 | 58 |
| | ACA | 7 | 14 | 3 |
| | ACG | 13 | 15 | 12 |
| Trp | TGG | 100 | 100 | 100 |
| Tyr | TAT | 6 | 26 | 33 |
| | TAC | 94 | 74 | 68 |
| Val | GTT | 6 | 7 | 40 |
| | GTC | 49 | 25 | 12 |
| | GTA | 3 | 5 | 20 |
| | GTG | 43 | 84 | 29 |

It should be noted that any combination of the above embodiments is within the scope of the invention. For example, the antigen-encoding sequence can be modified to lack a signal sequence and modified to reflect the human codon usage bias. Further, the antigen can be modified to have a heterologous signal sequence and further modified to reflect human codon usage bias. Further, the antigen-encoding sequence can be modified to lack a signal sequence and to be operably linked to a heterologous signal sequence.

### Antigen-encoding Polynucleotides For Use in the Invention

The polynucleotide vaccines of the invention can encode any antigen of interest, including allergens and antigens of pathogenic organisms (e.g., antigens of a bacterium, virus, parasite, fungus, yeast, and the like). As used herein, “antigen” is meant to encompass “allergens,” and thus antigen is used for clarity and without limitation herein unless specifically indicated otherwise. An “allergen” is generally meant to refer to an antigen that can produce a hypersensitivity reaction (allergy) in a sensitized subject.

Many antigens have been cloned and thus can be readily introduced into a construct for use as a polynucleotide vaccine in accordance with the invention. Where the antigens have not yet been cloned, methods are readily available in the art for obtaining nucleic acid encoding an antigen of interest, and manipulating the nucleic acid to provide a polynucleotide construct suitable for use in the invention.

Exemplary antigens for expression from a vaccine construct useful in the invention include, but are not necessarily limited to bacterial antigens (e.g., antigens of Bordetella spp. (e.g., B. pertussis), Mycobacteria spp. (e.g., M. tuberculosis, M. avium, M. intracellulare, M. kansaii, M. gordonae), Clostridia (e.g., C. tetani, C. perfringes and C. botulinum), Corynebacteria (e.g., C. diphtheria), Staphylcocci spp. (e.g., S. aureus, S. epidermidis), Streptococcus spp. (e.g., S. pyogenes (Group A Streptococcus), S. agalactiae (Group B Streptococcus), S. viridans, S. faecalis, S. bovis, anaerobic Streptococcus, S. pneumoniae), Bacillus spp. (e.g., B. anthracis), Hemophilus spp. (e.g., H. influenza), Neisseria spp. (e.g., N. gonorrhea, N. meningitidis), Pseudomonas spp. (e.g., P. aureginosa), pathogenic E. coli, (e.g., enteropathogenic, enterohemorrhagic, enteroinvasive, and enterotoxigenic E. coli), Salmonella spp. (e.g., S. typhi), Chlamyida spp. (e.g., C. trachomatis), and the like); Helicobacter pyloris, Borelia burgdorferi, Legionella pneumophilia, Listeria monocytogenes, pathogenic Campylobacter spp., Enterococcus sp., Erysipelothrix rhusiopathiae, Enterobacter aerogenes, Klebsiella pneumoniae, Pasturella multocida, Bacteroides spp., Fusobacterium nucleatum, Streptobacillus moniliformis, Treponema pallidium, Treponema pertenue, Leptospira, and Actinomyces spp. (e.g., A. israelli).

Further exemplary antigens suitable for use in the invention include, but are not limited to antigens of Retroviridae (e.g., human immunodeficiency viruses, such as HIV-1 (also referred to as HTLV-III, LAV or HTLV-III/LAV, or HIV-III; and other isolates (e.g., gp120 of HIV); Picomaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Bimaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), EBV, herpes viruses'); Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2-parenterally transmitted (i.e., Hepatitis C); Norwalk and related viruses, and astroviruses).

Exemplary antigens of pathogenic parasites that can be used in the invention include, but are not limited to Plasmodium (e.g., P. falciparum), Leishmania, Trypanosoma, Schistosoma, nematodes, cestodes, trematodes, and the like); fungal, yeast or other pathogen (e.g., Candida spp. (e.g., C. albcans), Pneumocystis carnii, Cryptococcus neoformans, Histoplasma capsulatum, Coccidioides immitis, Blastomyces dermatitidis, Candida albicans, and the like.

Viral antigens include, but are not necessarily limited to, capsid or core proteins (e.g., gag proteins, nucleocapsid proteins, polymerase, and the like), as well as envelope proteins and other viral proteins that may be suitable for use in eliciting an immune response in a host. Bacterial antigens include, but are not necessarily limited to, antigens of bacterial toxins (e.g., tetanus toxin, cholera toxin, diptheria toxin, toxins of pathogenic E. Coli, (heat labile toxin, heat stable toxin, and the like).

Where the vaccine is to be used as in allergic immunotherapy, the antigen is generally an allergen, e.g., an asthma-initiating allergen (see, e.g., U.S. Pat. Nos. 6,174,872 and 5,849, 719). In allergic asthma, the symptoms of the disease are triggered by an allergic response in a host to an allergen. The polynucleotide sequences of many nucleic acids which code for asthma-initiating antigen allergens are known. All such polynucleotide sequences are useful in the method of the invention. Examples of some of the more common allergens for use in the invention are set forth below; those of ordinary skill in the art will be familiar with additional examples, the use of which is encompassed by the invention.

Where the antigen is an allergen, nucleic acid encoding any of a variety of allergens are appropriate for use in the compositions of the invention. Non-limiting examples of known asthma-initiating antigen-encoding polynucleotides include those which code for IgE reactive major dust mite asthma-initiating antigens Der pI and Der pII (see, Chua, et al., J. Exp. Med., 167:175-182, 1988; and, Chua, et al., Int. Arch. Allergy Appl. Immunol., 91:124-129, 1990), the Der pII asthma-initiating antigen (see, Joost van Neerven, et al., J. Immunol., 151:2326-2335, 1993), the highly abundant Antigen E (Amb a1, includgin Amb a1.1, a1.2, and a1.3), ragweed pollen asthma-initiating antigen (see, Rafnar, eE al., J. Biol. Chem., 266:1229-1236, 1991), phospholipase A.sub.2 (bee venom) asthma-initiating antigen (see, Dhillon, et al., J. Allergy Clin. Immunol., 90:42-51, 1992), white birch pollen (Betvl) (see, Breiteneder, et al., EMBO, 8:1935-1938, 1989), and the Fel dI major domestic cat asthma-initiating antigen (see, Rogers, et al., Mol. Immunol., 30:559-568, 1993). Other allergens of interest include, but are not necessarily limited to, grass pollen or ragweed, latex, cat dander, food (such as peanut), house dust mite or cockroach allergen. The published sequences and methods for their isolation and synthesis described in these articles are incorporated herein by this reference to illustrate knowledge in the art regarding asthma-initiating antigen-encoding polynucleotides.

Allergens of interest, include, but are not limited to, ragweed pollen allergen Antigen E (Amb a1) (Rafnar et al. (1991) J. Biol. Chem. 266:1229-1236), major dust mite allergens DerpI and Der PII (Chua et al. (1988) J. Exp. Med. 167:175-182; Chua et al. (1990) Int. Arch. Allergy Appl. Immunol. 91:124-129), white birch pollen Bet vl (Breiteneder et al. (1989) EMBO J. 8:1935-1938), domestic cat allergen Fel dI (Rogers et al. (1993) Mol. Immunol. 30:559-568), and protein antigens from tree pollen (Elsayed et al. (1991) Scand. J. Clin. Lab. Invest. Suppl. 204:17-31). As indicated, allergens from trees are known, including allergens from birch, juniper and Japanese cedar. As Table 1 below indicates, in some embodiments, the allergen is a food allergen such as peanut allergen, for example Ara h I, and in some embodiments, the allergen is a grass allergen such as a rye allergen, for example Lol p I.

Other non-limiting examples of allergens include pollens, insect venoms, animal dander dust, fungal spores and drugs (e.g. penicillin). Examples of natural, animal and plant allergens include proteins specific to the following genera: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Altemaria (Altemaria altemata); Alder; Alnus (Alnus gultinosa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus anzonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

Further non-limiting examples of allergens are provided the table below, and further described in, e.g., PCT Publication No. WO 00/16804.

TABLE 1
Exemplary Allergens

| Group | Allergen | Reference |
|---|---|---|
| CRUSTACEA | | |
| Shrimp/lobster | tropomyosin | Leung et al. J. Allergy Clin. |
| | | Immunol., 1996, 98: 954-961 |
| | Pan s I | Leung et al. Mol. Mar. Biol. Bio- |
| | | technol., 1998, 7: 12-20 |
| INSECTS | | |
| Ant | Sol i 2 (venom) | Schmidt et al. J Allergy Clin |
| | | Immunol., 1996, 98: 82-8 |
| Bee | phospholipase A2 | Muller et al. J Allergy Clin Immunol, |
| | (PLA) | 1995, 96: 395-402 |
| | | Forster et al. J Allergy Clin Immunol, |
| | | 1995, 95: 1229-35 |
| | | Muller et al. Clin Exp Allergy, 1997, |
| | | 27: 915-20 |
| | Hyaluronidase | Soldatova et al. J Allergy Clin |
| | (Hya) | Immunol, 1998, 101: 691-8 |
| Cockroach | Bla g Bd9OK | Helm et al. J Allergy Clin Immunol, |
| | | 1996, 98: 172-80 |
| | Bla g 4 (a | Vailes et al. J Allergy Clin Immunol, |
| | calycin) | 1998, 101: 274-80 |
| | glutathione | Arruda et al. J Biol Chem, 1997, 272: |
| | S-transferase | 20907-12 |
| | Per a 3 | Wu et al. Mol Immunol, 1997, 34: |
| | | 1-8 |
| Dust mite | Der p 2 (major | Lynch et al. J Allergy Clin Immunol, |
| | allergen) | 1998, 101: 562-4 |
| | | Hakkaart et al. Clin Exp Allergy, |
| | | 1998, 28: 169-74 |
| | | Hakkaart et al. Clin Exp Allergy, |
| | | 1998, 28: 45-52 |
| | | Hakkaart et al. Int Arch Allergy |
| | | Immunol, 1998, 115 (2): 150-6 |
| | | Mueller et al. J Biol Chem, |
| | | 1997, 272: 26893-8 |
| | Der p 2 variant | Smith et al. J Allergy Clin Immunol, |
| | | 1998, 101: 423-5 |
| | Der f 2 | Yasue et al. Clin Exp Immunol, |
| | | 1998, 113: 1-9 |
| | | Yasue et al. Cell Immunol, 1997, |
| | | 181: 30-7 |
| | Der p 10 | Asturias et al. Biochim Biophys Acta, |
| | | 1998, 1397: 27-30 |
| | Tyr p 2 | Eriksson et al. Eur J Biochem, 1998 |
| Hornet | Antigen 5 aka | Tomalski et al. Arch Insect Biochem |
| | Dol m V | Physiol, 1993, 22: 303-13 |
| | (venom) | |
| Mosquito | Aed a I (salivary | Xu et al. Int Arch Allergy Immunol, |
| | apyrase) | 1998, 115: 245-51 |
| Yellow | antigen 5, | King et al. J Allergy Clin Immunol, |
| jacket | hyaluronidase, | 1996, 98: 588-600 |
| | and | |
| | phospholipase | |
| | (venom) | |
| MAMMALS | | |
| Cat | Fel d I | Slunt et al. J Allergy Clin Immunol, |
| | | 1995, 95: 1221-8 |
| | | Hoffmann et al. J Allergy Clin |
| | | Immunol, 1997, 99: 227-32 |
| | | Hedlin Curr Opin Pediatr, 1995, 7: |
| | | 676-82 |
| Cow | Bos d 2 (dander; | Zeiler et al. J Allergy Clin Immunol, |
| | a lipocalin) | 1997, 100: 721-7 |
| | | Rautiainen et al. Biochem Bioph. Res |
| | | Comm., 1998, 247: 746-50 |
| | β-lactoglobulin | Chatel et al. Mol Immunol, 1996, |
| | (BLG, major cow | 33: 1113-8 |
| | milk allergen) | Lehrer et al. Crit Rev Food Sci Nutr, |
| | | 1996, 36: 553-64 |
| Dog | Can f I and | Konieczny et al. Immunology, 1997, |
| | Can f 2, salivary | 92: 577-86 |
| | lipocalins | Spitzauer et al. J Allergy Clin |
| | | Immunol, 1994, 93: 614-27 |
| | | Vrtala et al. J Immunol, 1998, 160: |
| | | 6137-44 |
| Horse | Equ c 1 (major | Gregoire et al. J Biol Chem, 1996, |
| | allergen, | 271: 32951-9 |
| | a lipocalin) | |
| Mouse | mouse urinary | Konieczny et al. Immunology, 1997, |
| | protein | 92: 577-86 |
| | (MUP) | |

OTHER MAMMALIAN ALLERGENS

| | | |
|---|---|---|
| Insulin | | Ganz et al. J Allergy Clin Immunol, |
| | | 1990, 86: 45-51 |
| | | Grammer et al. J Lab Clin Med, |
| | | 1987, 109: 141-6 |
| | | Gonzalo et al. Allergy, 1998, 53: |
| | | 106-7 |
| Interferons | interferon alpha | Detmar et al. Contact Dermatis, |
| | 2c | 1989, 20: 149-50 |
| MOLLUSC | topomyosin | Leung et al. J Allergy Clin Immunol, |
| | | 1996, 98: 954-61 |

PLANT ALLERGENS:

| | | |
|---|---|---|
| Barley | Hor v 9 | Astwood et al. Adv Exp Med Biol, |
| | | 1996, 409: 269-77 |
| Birch | pollen allergen, | Twardosz et al. Biochem Bioph. Res |
| | Bet v 4 | Comm., 1997, 23 9: 197 |
| | rBet v 1 Bet v 2: | Pauli et al. J Allergy Clin Immunol, |
| | (profilin) | 1996, 97: 1100-9 |
| | | van Neerven et al. Clin Exp Allergy, |
| | | 1998, 28: 423-33 |
| | | Jahn-Schmid et al. Immunotech- |
| | | nology, 1996, 2: 103-13 |
| | | Breitwieser et al. Biotechniques, |
| | | 1996, 21: 918-25 |
| | | Fuchs et al. J Allergy Clin Immunol, |
| | | 1997, 100: 356-64 |
| Brazil | globulin | Bartolome et al. Allergol |
| nut | | Immunopathol, 1997, 25: 135-44 |
| Cherry | Pru a I (major | Scheurer et al. Mol Immunol, 1997, |
| | allergen) | 34: 619-29 |
| Corn | Zml 3 (pollen) | Heiss et al. FEBS Lett, 1996, 381: |
| | | 217-21 |
| | | Lehrer et al. Int Arch Allergy |
| | | Immunol, 1997, 113: 122-4 |
| Grass | Phl p 1, Phl p 2, | Bufe et al. Am J Respir Crit Care |
| | Phl p 5 | Med, 1998, 157: 1269-76 |
| | (timothy grass | Vrtala et al. J Immunol Jun 15, 1998, |
| | pollen) | 160: 6137-44 |
| | | Niederberger et al. J Allergy Clin |
| | | Immun., 1998, 101: 258-64 |
| | Hol 1 5 velvet | Schramm et al. Eur J Biochem, 1998, |
| | grass pollen | 252: 200-6 |
| | Bluegrass | Zhang et al. J Immunol, 1993, 151: |
| | allergen | 791-9 |
| | Cyn d 7 Bermuda | Smith et al. Int Arch Allergy |
| | grass | Immunol, 1997, 114: 265-71 |
| | Cyn d 12 (a | Asturias et al. Clin Exp Allergy, |
| | profilin) | 1997, 27: 1307-13 |
| | | Fuchs et al. J Allergy Clin Immunol, |
| | | 1997, 100: 356-64 |
| Juniper | Jun o 2 (pollen) | Tinghino et al. J Allergy Clin |
| | | Immunol, 1998, 101: 772-7 |
| Latex | Hev b 7 | Sowka et al. Eur J Biochem, 1998, |
| | | 255: 213-9 |
| | | Fuchs et al. J Allergy Clin Immunol, |
| | | 1997, 100: 356-64 |
| Mercurialis | Mer a I (profilin) | Vallverdu et al. J Allergy Clin |
| | | Immunol, 1998, 101: 363-70 |
| Mustard | Sin a I (seed) | Gonzalez de la Pena et al. Biochem |
| (Yellow) | | Bioph. Res Comm., 1993, 190: |
| | | 648-53 |
| Oilseed | Bra r I pollen | Smith et al. Int Arch Allergy |
| rape | allergen | Immunol, 1997, 114: 265-71 |
| Peanut | Ara h I | Stanley et al. Adv Exp Med Biol, |
| | | 1996, 409: 213-6 |
| | | Burks et al. J Clin Invest, 1995, |
| | | 96: 1715-21 |
| | | Burks et al. Int Arch Allergy |
| | | Immunol, 1995, 107: 248-50 |
| Poa | Poa p 9 | Parronchi et al. Eur J Immunol, |
| pratensis | | 1996, 26: 697-703 |
| | | Astwood et al. Adv Exp Med Biol, |
| | | 1996, 409: 269-77 |
| Ragweed | Amb a I | Sun et al. Biotechnology Aug, 1995, |
| | | 13: 779-86 |
| | | Hirschwehr et al. J Allergy Clin |
| | | Immunol, 1998, 101: 196-206 |
| | | Casale et al. J Allergy Clin Immunol, |
| | | 1997, 100: 110-21 |
| Rye | Lol p I | Tamborini et al. Eur J Biochem, |
| | | 1997, 249: 886-94 |
| Walnut | Jug r I | Teuber et al. J Allergy Clin Immun., |
| | | 1998, 101: 807-14 |
| Wheat | allergen | Fuchs et al. J Allergy Clin Immunol, |
| | | 1997, 100: 356-64 |
| | | Donovan et al. Electrophoresis, 1993, |
| | | 14: 917-22 |
| FUNGI: | | |
| Aspergillus | Asp f 1, Asp f 2, | Crameri et al. Mycoses, 1998, 41 |
| | Asp f 3, Asp f 4, | Suppl 1: 56-60 |
| | rasp f 6 | Hemmann et al. Eur J Immunol, |
| | | 1998, 28: 1155-60 |
| | | Banerjee et al. J Allergy Clin |
| | | Immunol, 1997, 99: 821-7 |
| | | Crameri Int Arch Allergy Immunol, |
| | | 1998, 115: 99-114 |
| | | Crameri et al. Adv Exp Med Biol, |
| | | 1996, 409: 111-6 |
| | | Moser et al. J Allergy Clin Immunol, |
| | | 1994, 93: 1-11 |
| | Manganese | Mayer et al. Int Arch Allergy |
| | superoxide | Immunol, 1997, 113: 213-5 |
| | dismutase | |
| | (MNSOD) | |
| Blomia | allergen | Caraballo et al. Adv Exp Med Biol, |
| | | 1996, 409: 81-3 |
| Penicillinium | allergen | Shen et al. Clin Exp Allergy, 1997, |
| | | 27: 682-90 |
| Psilocybe | Psi c 2 | Horner et al. Int Arch Allergy |
| | | Immunol, 1995, 107: 298-300 |

The recombinant expression vector component of the polynucleotide compositions of the invention may encode one or more antigens, different or multiple copies of the same antigenic or immunogenic peptides of antigens, or a combinations thereof. Many antigen-encoding polynucleotides are known in the art; others can be identified using conventional techniques.

### Administration and Dosage

The polynucleotide vaccines of the invention are administered to an individual using any available method and route suitable for drug delivery, including systemic, mucosal, and localized routes of administration. In a preferred embodiment of the method, the polynucleotide vaccine is administered via a systemic, enteral or topical route. Examples of systemic routes include, but are not limited to, intradermal, intramuscular, subcutaneous and intravenous administration. Examples of topical routes include, but are not limited to, intranasal, intravaginal, intrarectal, intratracheal, transdermal and ophthalmic administration. Examples of enteral routes include, but are not limited to, oral and gastric administration. Routes of administration may be combined, if desired, or adjusted depending upon the construct, the number of ISS, the desired effect on the immune response, and other variables that will be readily apparent to the ordinarily skilled artisan. In general, the immunomodulatory nucleic acid can be administered as part of the polynucleotide vaccine (e.g., within the same nucleic acid molecule), or as a separate nucleic acid molecule that is co-administered or separately administered.

The polynucleotide construct composition can be administered in a single dose or in multiple doses, and may encompass administration of booster doses, to elicit and/or maintain the desired effect on the immune response. The local activation of innate immunity will enhance the protective effect of the vaccine. Topical administration can also avoid unwanted side effects caused by systemic administration.

Treatment includes prophylaxis and therapy. Prophylaxis or therapy can be accomplished by a single direct administration at a single time point or multiple time points. Administration can also be delivered to a single or to multiple sites.

The subject can be any vertebrate, but will preferably be a mammal. Mammals include human, bovine, equine, canine, feline, porcine, and ovine animals. If a mammal, the subject will preferably be a human, but may also be a domestic livestock, laboratory subject or pet animal.

Inhalational routes of administration (e.g., intranasal, intrapulmonary, and the like) are particularly useful in modulation of allergic responses. Inhalational delivery can be accomplished by inhalation of aerosol suspensions or insufflation of the polynucleotide compositions of the invention. Nebulizer devices, metered dose inhalers, and the like suitable for delivery of polynucleotide compositions to the nasal mucosa, trachea and bronchioli are well-known in the art and will therefore not be described in detail here. For general review in regard to intranasal drug delivery, see, e.g., Chien, Novel Drug Delivery Systems, Ch. 5 (Marcel Dekker, 1992).

Parenteral routes of administration other than inhalation administration include, but are not necessarily limited to, topical, transdermal, subcutaneous, intramuscular, intraorbital, intracapsular, intraspinal, intrastemal, and intravenous routes, i.e., any route of administration other than through the alimentary canal. Parenteral administration can be carried to effect systemic or local delivery of the polynucleotide formulations of the invention. Where systemic delivery is desired, administration typically involves invasive or systemically absorbed topical or mucosal administration of pharmaceutical preparations.

The constructs of the invention can also be delivered to the subject by enteral administration. Enteral routes of administration include, but are not necessarily limited to, oral and rectal (e.g., using a suppository) delivery.

Methods of administration of the constructs of the invention by administration through the skin or mucosa include, but are not necessarily limited to, topical application of a suitable pharmaceutical preparation, transdermal transmission, injection and epidermal administration. For transdermal transmission, absorption promoters or iontophoresis are suitable methods. For review regarding such methods, those of ordinary skill in the art may wish to consult Chien, supra at Ch. 7. Iontophoretic transmission may be accomplished using commercially available “patches” which deliver their product continuously via electric pulses through unbroken skin for periods of several days or more. An exemplary patch product for use in this method is the LECTRO PATCH™ (manufactured by General Medical Company, Los Angeles, Calif.) which electronically maintains reservoir electrodes at neutral pH and can be adapted to provide dosages of differing concentrations, to dose continuously and/or to dose periodically.

Epidermal administration can be accomplished by mechanically or chemically irritating the outermost layer of the epidermis sufficiently to provoke an immune response to the irritant. An exemplary device for use in epidermal administration employs a multiplicity of very narrow diameter, short tynes which can be used to scratch polynucleotide composition coated onto the tynes into the skin. The device included in the MONO-VACC™ tuberculin test (manufactured by Pasteur Merieux, Lyon, France) is suitable for use in epidermal administration of the polynucleotides of the invention.

The invention also contemplates opthalmic administration, which generally involves invasive or topical application of a pharmaceutical preparation to the eye. Eye drops, topical cremes and injectable liquids are all examples of suitable formulations for delivering drugs to the eye.

Where the antigen is of a pathogenic organism, the polynucleotide vaccines of the invention can be administered to a subject prior to or after exposure to a pathogenic antigen (e.g., prior to or after exposure to the pathogen), but preferably prior to onset of disease symptoms associated with infection. In some embodiments, the polynucleotide vaccines are administered after infection is established and/or after onset of disease symptoms. As such, such polynucleotide vaccine compositions can be administered at any time after exposure to the pathogen, but a first dose is usually administered about 8 hours, about 12 hours, about 24 hours, about 2 days, about 4 days, about 8 days, about 16 days, about 30 days or 1 month, about 2 months, about 4 months, about 8 months, or about 1 year after exposure. The invention also provides for administration of subsequent doses of the polynucleotide vaccine.

### Dosages

One particular advantage of the use of compositions of the invention is that the vaccines and immunomodulatory nucleic acid molecules exert immunomodulatory activity even at relatively low dosages. Although the dosage used will vary depending on the clinical goals to be achieved, a suitable dosage range is one which provides up to about 1 μg, to about 1,000 μg, to about 10,000 μg, to about 25,000 μg or about 50,000 μg of nucleic acid of the polynucleotide composition. The polynucleotide vaccines can be administered in a single dosage or several smaller dosages over time.

Based on current studies, immunomodulatory nucleic acid molecules are believed to have little or no toxicity at these dosage levels.

Where the antigen-encoding nucleic acid and the ISS are administered as separate components, the relative amounts of antigen-encoding nucleic acid and ISS can be varied. For example, the ratio of ISS to antigen-encoding nucleic acid (by weight) can be about 1:125 (e.g., 0.4 μg:50 μg), 1:100, 1:50, 1:25 (e.g., 2 μg:50 μg), 1:10, 1:5, 1:1 or any other suitable weight:weight ratio. The molar ratio of ISS polynucleotide to antigen-encoding polynucleotide can range from about 1.25:1 to about 100:1; from about 1.5:1 to about 50:1; from about 5:1 to about 25:1, from about 10:1 to about 20:1, and can be about 2:1, about 2.5:1, about 3:1, about 4:1; about 5:1, about 5.5: 1; or about 15:1. In general, the higher the expression level of the antigen from the antigen-encoding polynucleotide of the vaccine composition, the more ISS can be included in the composition as a adjuvant without adversely affecting the expression (e.g., without suppressing antigen expression). Generally, ISS and antigen-encoding polynucleotide are administered from about 0.5 mg to about 5 mg each, and can be provided at a 1:1 weight ratio.

It should be noted that the immunotherapeutic activity of immunomodulatory nucleic acid molecules, as well as the polynucleotide vaccines, is essentially dose-dependent. Therefore, to increase ISS potency by a magnitude of two, each single dose is doubled in concentration. Increased dosages may be needed to achieve the desired therapeutic goal. The invention thus contemplates administration of “booster” doses to provide and maintain an immune response effective to, for example, protect the subject from infection or to inhibit infection; to reduce the risk of the onset of disease or the severity of disease symptoms that may occur as a result of infection; to facilitate reduction of pathogen load; to facilitate clearance of infecting pathogen from the subject (e.g., to facilitate clearance of organisms from the lungs), and the like; or to maintain the resistance of the subject to exposure to allergen (e.g., to protect the subject from a hypersensitivity reaction, e.g., an early or late phase allergic response, including anaphylaxis).

When multiple doses are administered, subsequent doses are administered within about 16 weeks, about 12 weeks, about 8 weeks, about 6 weeks, about 4 weeks, about 2 weeks, about 1 week, about 5 days, about 72 hours, about 48 hours, about 24 hours, about 12 hours, about 8 hours, about 4 hours, or about 2 hours or less of the previous dose. In one embodiment, ISS are administered at intervals ranging from at least every two weeks to every four weeks (e.g., monthly intervals) in order to maintain the maximal immune response.

In view of the teaching provided by this disclosure, those of ordinary skill in the clinical arts will be familiar with, or can readily ascertain, suitable parameters for administration of ISS according to the invention.

### Formulations

The invention provides compositions, both prophylactic and therapeutic, comprising a polynucleotide vaccine and/or an ISS of the invention. Such compositions optionally include a pharmaceutically acceptable carrier. The polynucleotide vaccine and/or ISS of the invention can be prepared in a variety of formulations, including conventional pharmaceutically acceptable carriers, and, for example, microbeads, microspheres, capsules designed for oral delivery, etc. The ISS can optionally be administered in conjunction with a drug useful in the treatment of the subject's condition. Such additional agents can be administered separately or included in the polynucleotide vaccine and/or ISS composition.

In general, polynucleotide vaccine compositions are prepared in a pharmaceutically acceptable composition for delivery to a host. Pharmaceutically acceptable carriers preferred for use with the polynucleotides may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A polynucleotide composition may also be lyophilized using means well known in the art, for subsequent reconstitution and use according to the invention. Also of interest are formulations for liposomal delivery, and formulations comprising microencapsulated polynucleotides.

In general, the pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions comprising the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. Preservatives and other additives may also be present such as, for example, antimicrobial agents (e.g., antimicrobials, antibacterials, antivirals, antifungals, etc.), antioxidants, chelating agents, and inert gases and the like.

The polynucleotide vaccine compositions can be administered in the absence of agents or compounds that might facilitate uptake by target cells (e.g., as a “naked” polynucleotide, e.g., a polynucleotide that is not encapsulated by a viral particle). Polynucleotide vaccine compositions can also be administered with compounds that facilitate uptake of polynucleotides by target cells (e.g., by macrophages) or otherwise enhance transport of the polynucleotides to a treatment site for action. Absorption promoters, detergents and chemical irritants (e.g., keratinolytic agents) can enhance transmission of a polynucleotide vaccine composition into a target tissue (e.g., through the skin). For general principles regarding absorption promoters and detergents which have been used with success in mucosal delivery of organic and peptide-based drugs, see, e.g., Chien, Novel Drug Delivery Systems, Ch. 4 (Marcel Dekker, 1992). Examples of suitable nasal absorption promoters in particular are set forth at Chien, supra at Ch. 5, Tables 2 and 3; milder agents are preferred. Suitable agents for use in the method of this invention for mucosal/nasal delivery are also described in Chang, et al., Nasal Drug Delivery, “Treatise on Controlled Drug Delivery”, Ch. 9 and Tables 3-4B thereof, (Marcel Dekker, 1992). Suitable agents which are known to enhance absorption of drugs through skin are described in Sloan, Use of Solubility Parameters from Regular Solution Theory to Describe Partitioning-Driven Processes, Ch. 5, “Prodrugs: Topical and Ocular Drug Delivery” (Marcel Dekker, 1992), and at places elsewhere in the text. All of these references are incorporated herein for the sole purpose of illustrating the level of knowledge and skill in the art concerning drug delivery techniques.

A colloidal dispersion system may be used for targeted delivery of a polynucleotide vaccine composition to specific tissue. Colloidal dispersion systems include macromolecule complexes, nanocapsules, microspheres, beads, and lipid-based systems including oil-in-water emulsions, micelles, mixed micelles, and liposomes.

Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA and DNA can be encapsulated within the aqueous interior and be delivered to cells in a biologically active form (Fraley, et al, (1981) Trends Biochem. Sci., 6:77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

While not necessary, where desired targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

While not necessary, the surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various well known linking groups can be used for joining the lipid chains to the targeting ligand (see, e.g., Yanagawa, et al., (1988) Nuc. Acids Symp. Ser., 19:189; Grabarek, et al., (1990) Anal. Biochem., 185:131; Staros, et al., (1986) Anal. Biochem. 156:220 and Boujrad, et al., (1993) Proc. Natl. Acad. Sci. USA, 90:5728). Targeted delivery of polynucleotides of the polynucleotide vaccine composition can also be achieved by conjugation of the polynucleotide to the surface of viral and non-viral recombinant expression vectors, to an antigen or other ligand, to a monoclonal antibody or to any molecule which has the desired binding specificity.

In view of the teaching provided by this disclosure, those of ordinary skill in the clinical arts will be familiar with, or can readily ascertain, suitable parameters for administration of the polynucleotide vaccine compositions according to the invention, including combination of polynucleotide vaccine composition administration with conventional treatments.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

### Expression of Amb A1 in COS-7 Cells

This example demonstrates the enhancement of antigen expression from a polynucleotide vector following progressive modifications. The coding sequence for the ragweed allergen Amb a1 was cloned into the pNDKm vector. The PNDKm vector was generated from the pND vector (gift of G. Rhode), by replacement of the AmpR gene with the KanR gene, thereby removing two ISS segments. Thus, the pDNKm vector lacks the two most potent ISS sequences (AACGTT).

Ragweed allergen Amb a1 has been previously cloned and sequenced (see, e.g., Rafnar ewt al. J. Biol. Chem. 266, 1229-1236 (1991); GenBank Accession Nos. M63116, M80558, M62981, M80559, M62961, and M80560, as well as Griffith, et al. Int. Arch. Allergy Appl. Immunol. 96, 296-304 (1991), and GenBank Accession No. M80562). The sequence of Amb a1.1 was used the constructs described in the present examples, and is referred to as Amb a1 for ease in reference.

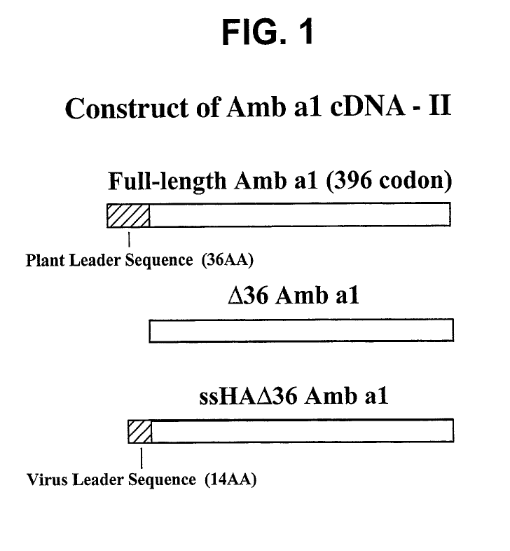

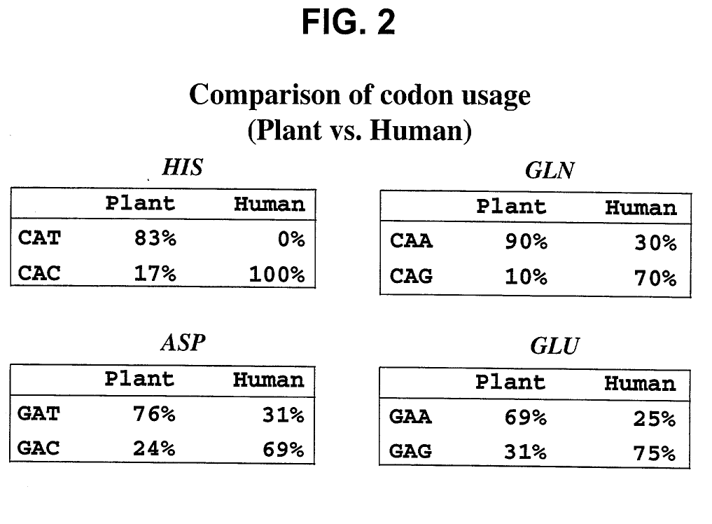

The pNKDm Amb a1 construct (the Amb a1 coding sequence in the pNDKm vector, referred to as the Amb a1/pNKDm construct) was modified to delete the Amb a1 36 amino acid signal sequence using methods well known in the art, to create the construct referred to as Δ36Amb a1/pNKDm (see FIG. 1 for a schematic). The signal sequence of influenza virus hemagglutinin A (HA) was then operably positioned upstream of the Amb a1 coding sequence to create the construct ssHAΔ36Amb a1 (see FIG. 1). An Amb a1 sequence was altered using methods according to the art to create an Amb a1 having a human coding usage bias, creating the construct hssHAΔ36Amb a1 (see FIG. 1). The codons of Amb a1 which can be altered to reflect the codon usage bias in humans are illustrated in FIG. 2. The sequence of altered Amb al (HssHAΔ36Amb a1), is provided in the Sequence Listing (SEQ ID NO:3).

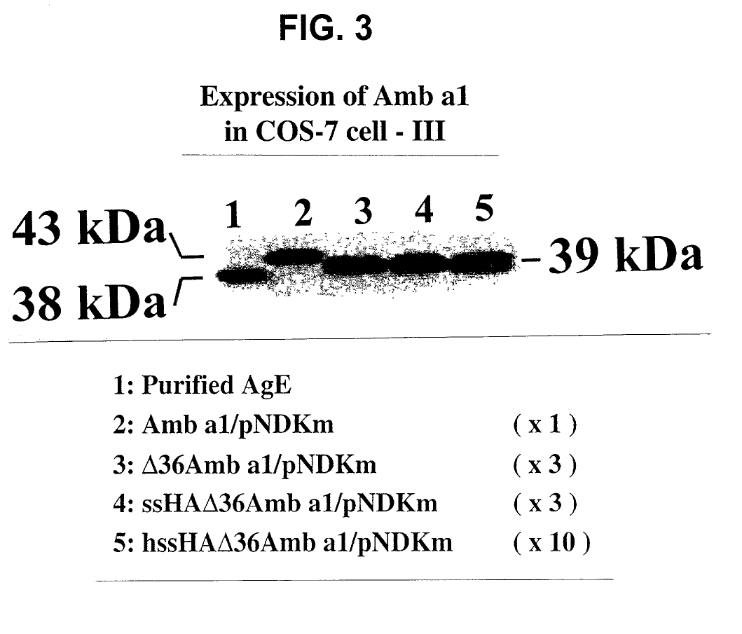

Each of the above constructs was introduced into COS-7 cells to analyze their relative expression levels. A Western blot showing expression of Amb a1 in COS-7 cells using various plasmids is presented in FIG. 3. Lane 1 shows purified Amb a1 (also referred to as Antigen E or AgE) as a control. Lane 2 shows the baseline expression with unmodified Amb a1 in COS-7 cells from a pNDKm vector. Lane 3 shows about a 3-fold increase in expression over baseline using the vector of lane 2 modified to delete the plant leader sequence (Δ36Amb a1/pNDKm). Lane 4 shows also about a 3-fold expression increase over baseline (lane 2) for the same vector with a hemagglutinin signal sequence added (ssHAΔ36Amb a1/pNKDm). Lane 5 shows about a 10-fold increase in expression over baseline using the same vector as in lane 4, but with a humanized codon bias (hssHAΔ36Amb a1/pNDKm).

### Example 2

### Induction of Antigen-specific Antibody and Cytokine in Vivo

This example demonstrates that the antigen expressed by modified polynucleotide vaccines in accordance with the invention is capable of inducing significantly enhanced immune responses in an antigen-specific manner.

Mice were immunized by intradermal injection at the base of the tail 3 times, 2 weeks apart, with one of the following plasmids: pNDKm (control); Amb a1/pNDKm (containing Amb a1); Δ36pNDKmAmb a1/pNDKm (containing Amb a1 with 36 amino acid signal sequence deleted); ssHAΔ36pNDKmAmb a1/pNDKm (containing Amb a1 modified to substitute a hemagglutinin A signal sequence for the native Amb a1 36 amino acid leader sequence); or hssHAΔ36pNDKmAmb a1/pNDKm (ssHAΔ36pNDKmAmb a1/pNDKm with a human codon bias; see FIG. 2).

Blood samples were drawn at the time of immunization, and at 2, 4 and 6 weeks post-immunization, and anti-Amb a1 IgG2a antibody levels determined by routine ELISA techniques. To study IFN-γ production, the mice were sacrificed at 6 weeks after immunization, splenocytes incubated with anti-CD3 and anti-CD28 antibodies in vitro for 24 hrs, and these supernatants were assayed for the presence of IFN-γ by sandwich ELISA (Martin-Orozco, et al. (1999) Int. Immun. 11:1111-1118).

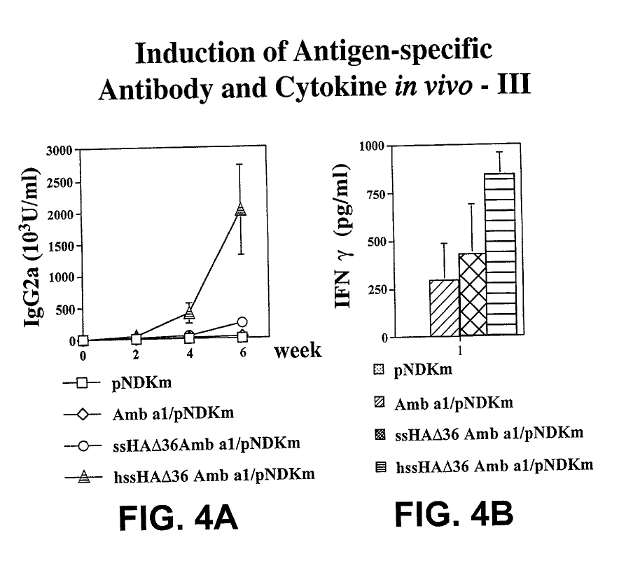

Amb a1-specific IgG2a levels, in 10³ U/ml, in mice 0, 2, 4 and 6 weeks after immunization with plasmid are shown in FIG. 4A. Amb a1-specific interferon gamma (IFNγ), in pg/ml, released in vitro by CD4+ T cells of immunized mice is shown in FIG. 4B. Results shown in the figures are means, plus or minus standard error of the mean, for at least 4 animals per group.

### Example 3

### Induction of Antigen-specific Immune Responses With ISS

This example demonstrates the synergistic enhancement of the immune response to an antigen encoded by a polynucleotide vaccine modified in accordance with the invention and co-injected with ISS-ODN.

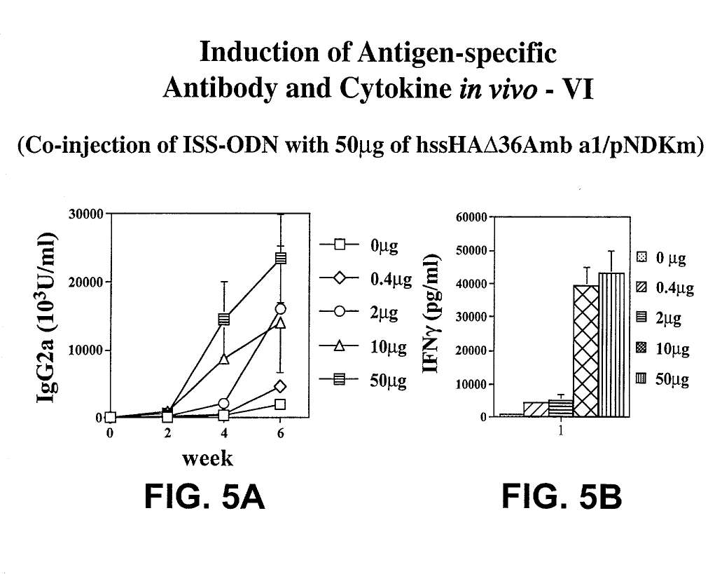

FIG. 5A is a line graph showing Amb a1-specific IgG2a levels, in 10³ U/ml, in mice 0, 2, 4 and 6 weeks after immunization with pNDKm containing Amb a1 modified to substitute a hemagglutinin A signal sequence for the native Amb a1 36 amino acid leader sequence and modified to humanize its codon bias. In addition to 50 μg of the hssHAΔ36Amb a1/pNDKm construct, the mice were co-injected with ISS-ODN in the following amounts: 0 μg (open squares), 0.4 μg (open diamonds), 2 μg (open circles), 10 μg (open triangles), or 50 μg (closed squares).

FIG. 5B is a bar graph showing levels of Amb a1-specific interferon gamma (IFNγ), in pg/ml, released in vitro by CD4+ T cells of mice 6 weeks after immunization with 50 μg of the hssHAΔ36Amb a1/pNDKm construct, and co-injected with ISS-ODN in the following amounts: 0 μg (first bar from left), 0.4 μg (second bar from left), 2 μg (third bar from left), 10 μg (fourth bar from left), or 50 μg (fifth bar from left).

### Example 4

### In Vivo Efficacy of Differing Forms of ISS-ODN

This example demonstrates increased immunostimulatory effects of ISS-ODN in single-stranded form, having a synthetic, sulfur-containing phosphorothioate backbone (ssPS), as compared to double-stranded form, having a native phosphodiester backbone (dsPO). In short, mice were injected i.v. with PBS (control) or 200 μg/mouse of dsPO ISS (5′-TGACTGTGAACGTTCGAGATGA-3′ (SEQ ID NO:1), control, dsPO mutated-ODN (M-ODN) was 5′-TGACTGTGAAGGTTAGAGATGA-3′ (SEQ ID NO:4), ssPS ISS (of the same sequence as the dsPO ISS), or ssPS M-ODN (of the same sequence as the dsPO M-ODN). Two weeks after injection, the mice were sacrificed, spleen cells isolated, and mRNA isolated from the spleen cells.

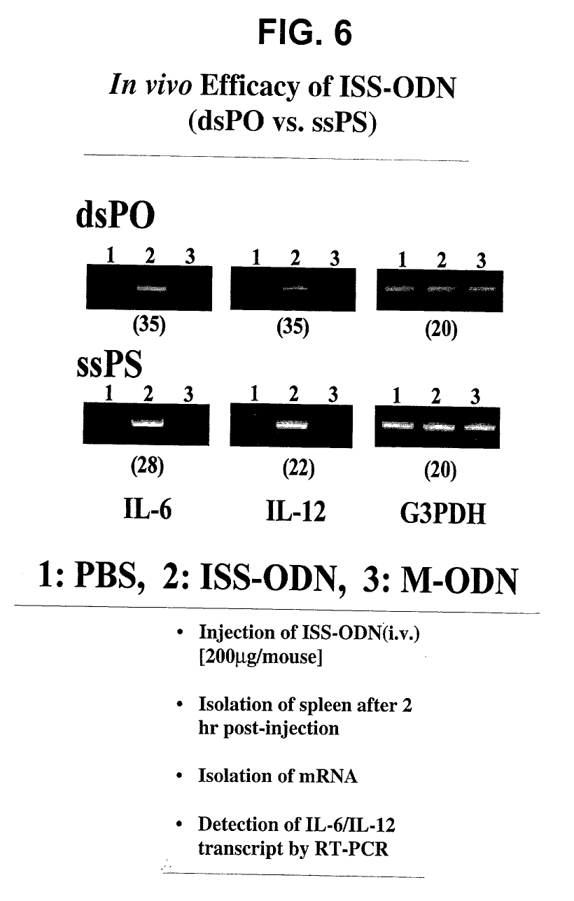

FIG. 6 shows transcripts of IL-6 (first column), IL-12 (second column), and G3PDH (third column) detected by RT-PCR in mRNA isolated from spleen of mice 2 hours after intravenous injection with 200 μg of ISS-ODN, either (dsPO) or (ssPS).

For each gel, lane 1 represents mice treated with phosphate buffered saline (PBS) in lieu of ISS-ODN, lane 2 represents mice treated with ISS-ODN, and lane 3 represents mice treated with a mutant version of the ODN that lacks immunostimulatory sequences.

### Example 5

### Amplification of Immune Responses Via Immunological Memory

This example demonstrates that vaccine efficacy can be amplified by taking advantage of immunological memory induced by prior exposure to an antigen. Based on this principle, limitations in the efficacy of polynucleotide vaccines can be overcome by including in the vaccine construct a universal antigen, one for which hosts receiving the vaccine will already have immunological memory.

Balb/c mice were primed intradermally with a β-galactosidase (β-gal) based gene vaccine (pCMVLacZ, 50 μg) 3 times, 2 weeks apart. Two months after the first immunization, mice were boosted once with ovalbumin (OVA, 4 μg) in alum, β-gal (10 μg) in alum, OVA and β-gal (4 μg and 10 μg, respectively) in alum, or with OVA conjugated to β-gal (OVA/β-gal molar ratio of 1; 4 μg of OVA conjugated to 10 μg of β-gal). The immune response to OVA was followed for the subsequent 6 weeks. Mice were then sacrificed and cytokine profile (IFNγ) and antibody titers to OVA were determined.

In this model, β-gal induces a memory response to enhance the primary response to OVA. The results are shown in Table 1, and indicate that the memory response to β-gal could be recruited to enhance the primary immune response to OVA only when the OVA antigen was fused to β-gal. Furthermore, despite the injection of OVA-β-gal conjugate in alum, the response to OVA had Th1 characteristics (IFNγ and IgG2a). The greater efficacy observed with conjugation of β-gal to OVA may be attributable to more balanced expression levels of β-gal and OVA when delivered in this conjugated form. Efficacy of OVA mixed with β-gal (not conjugated) may be improved by strategies that will result in more balanced expression.

TABLE 1

| Immu- | | IFNγ | Anti-OVA IgG1 | Anti-OVA |
|---|---|---|---|---|
| nization | Boosting | (pg/ml) | (Units) | IgG2a (Units) |
| pCMV- | β-gal | <50 | <2000 | <2000 |
| LacZ | | | | |
| pCMV- | OVA | <50 | 36,072 ± 4,154 | <2000 |
| LacZ | | | | |
| pCMV- | OVA | <50 | 28,189 ± 2,887 | 59,150 ± 24,500 |
| LacZ | mixed | | | |
| | with β- | | | |
| | gal | | | |
| pCMV- | OVA-β- | 223 ± 53 | 1,670,850 ± | 643,700 ± 53,578 |
| LacZ | gal | | 263,409 | |
| | conju- | | | |
| | gate | | | |

### Example 6

### Reduction of Amb A1-specific IgE by Administration of ISS and modified Amb A1 Expression Construct

This Example demonstrates that administration of ISS with a construct encoding an antigen modified to have a heterologous signal sequence and a human codon bias to an antigen-sensitized host results in reduction of antigen-specific IgE levels following subsequent challenge.

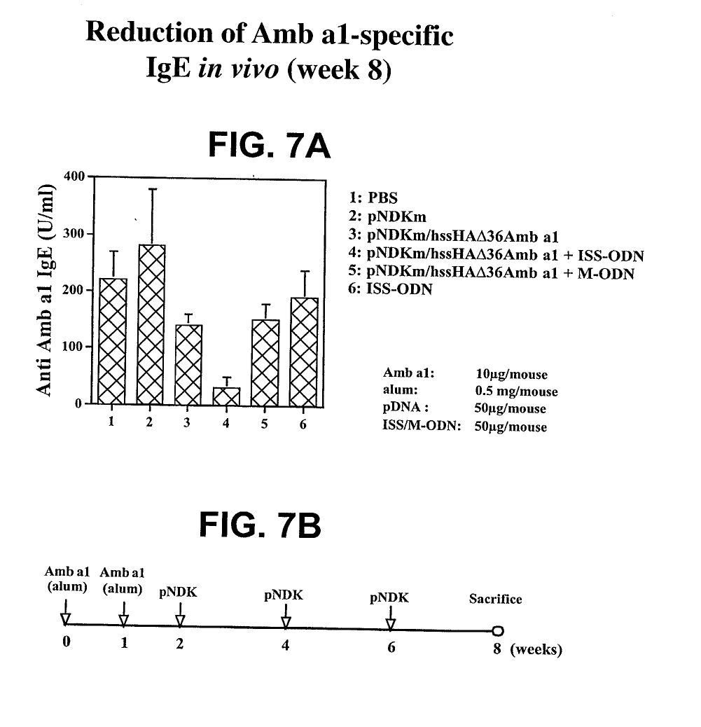

Mice were sensitized to Amb a1 by subcutaneous administration of Amb al (10 μg/mouse) admixed with alum (0.5mg/mouse), twice at one week intervals (see FIG. 7B for immunization schedule). At 2, 4 and 6 weeks after antigen administration, the mice received an intradermal administration of either 1) PBS (control), 2) pNDKm (control; 50 μg/mouse), 3) pNDKm/hssHAΔ36Amb a1 (Amb a1 modified to delete the native leader sequence, incorporate the HA signal sequence, and has human codon usage bias;; 50 μg/mouse); 4) pNDKm/hssHAΔ36Amb a1 (; 50 μg/mouse) co-administered with ISS (5′-TGACTGTGAACGTTCGAGATGA-3′ (SEQ ID NO:1); 50 μg/mouse); 5) pNDKm/hssHAΔ36Amb a1 (; 50 μg/mouse) co-administered with M-ODN (control; 5′-TGACTGTGAAGGTTAGAGATGA-3′ (SEQ ID NO:4); 50 μg/mouse); or 6) ISS alone (50 μg/mouse).

Mice were sacrificed at 8 weeks after sensitization, and Amb a1-specific IgE levels were detected by ELISA. The results are show in FIG. 7A.

Those skilled in the art will appreciate that the conceptions and specific embodiments disclosed in the foregoing description may be readily utilized as a basis for modifying or designing other embodiments for carrying out the same purposes of the present invention.

What is claimed is:

1. A polynucleotide vaccine comprising a nucleic acid sequence encoding an antigen derived from a non-host species of a first phylum or first kingdom, wherein the nucleic acid sequence encoding the antigen is modified by deletion of a native signal sequence.

2. The polynucleotide vaccine of claim 1, wherein the nucleic acid sequence encoding the antigen is further modified to include a signal sequence derived from a second phylum or second kingdom, wherein the signal sequence is operably linked to the antigen-encoding sequence.

3. The polynucleotide vaccine of claim 2, wherein the signal sequence comprises a hemagglutinin A (HA) signal sequence.

4. The polynucleotide vaccine of claim 1, wherein at least one codon of the nucleic acid sequence encoding the antigen is modified from a wild type sequence of the non-host species to an analogous codon of a host species.

5. The polynucleotide vaccine of claim 1, further comprising a universal antigen or an immunogenic fragment thereof.

6. The polynucleotide vaccine of claim 1, wherein the first kingdom is plant.

7. The polynucleotide vaccine of claim 1, wherein the antigen is Amb a1.

8. The polynucleotide vaccine of claim 1, wherein the antigen is derived from a pathogen.

9. The polynucleotide vaccine of claim 8, wherein the pathogen is a bacterium, a virus or a parasite.

10. A method for modulating an immune response to an antigen comprising administering to a subject a polynucleotide vaccine of any one of claims 1-9 in an amount effective to modulate an immune response to the antigen.

11. The method of claim 10, further comprising administering to the subject an immunostimulatory nucleotide sequence (ISS).

12. The method of claim 10, wherein the antigen is an allergen.

13. The method of claim 12, wherein the allergen is a plant, food, latex, cat dander, cockroach or house dust mite allergen.

14. The method of claim 13, wherein the plant allergen is ragweed or grass pollen.

15. A method for eliciting an immune response to an antigen comprising administering to a subject a polynucleotide vaccine of any one of claims 1-9 in an amount effective to elicit an immune response to the antigen.

16. The method of claim 15, further comprising administering to the subject an immunostimulatory nucleotide sequence (ISS).

17. The method of claim 15, wherein the antigen is derived from a pathogen.

18. The method of claim 17, wherein the pathogen is a bacterium, a virus or a parasite.

19. The method of claim 11, wherein the ISS comprises an unmethylated 5′-CG-3′ nucleotide sequence.

20. The method of claim 19, wherein the ISS comprises a sequence selected from the group consisting of: 5′-rrcgyy-3′, 5′-rycgyy-3′, 5′-rrcgyycg-3′, 5′-rycgyycg-3′ or 5′-(TCG)m-3′.

21. The method of claim 20, wherein the sequence is selected from the group consisting of: AACGTT, AGCGTT, GACGTT, GGCGTT,AACGTC, AGCGTC, GACGTC, GGCGTC, AACGCC, AGCGCC, GACGCC, GGCGCC, AACGCT, AGCGCT, GACGCT, GGCGCT, ATCGTT, ACCGTT, GTCGTT, GCCGTT, ATCGTC, ACCGTC, GTCGTC, GCCGTC, ATCGCT, ACCGCT, GTCGCT, GCCGCT, ATCGCC, ACCGCC, GTCGCC, GCCGCC, AACGTTCG, AGCGTTCG, GACGTTCG, GGCGTTCG, AACGTCCG, AGCGTCCG, GACGTCCG, GGCGTCCG, AACGCCCG, AGCGCCCG, GACGCCCG, GGCGCCCG, AACGCTCG, AGCGCTCG, GACGCTCG, GGCGCTCG, ATCGTTCG, ACCGTTCG, GTCGTTCG, GCCGTTCG, ATCGTCCG, ACCGTCCG, GTCGTCCG, GCCGTCCG, ATCGCTCG, ACCGCTCG, GTCGCTCG, GCCGCTCG, ATCGCCCG, ACCGCCCG, GTCGCCCG and GCCGCCCG.

22. A polynucleotide vaccine comprising a nucleic acid sequence encoding an Amb a1 allergen modified by deletion of a native Amb a1 signal sequence.

23. The polynucleotide vaccine of claim 22, wherein the nucleic acid sequence encoding the Amb a1 allergen is further modified to comprise a heterologous signal sequence operably linked to the Amb a1 allergen-encoding sequence.

24. The polynucleotide vaccine of claim 23, wherein the heterologous signal sequence comprises a hemagglutinin A (HA) signal sequence.

25. The polynucleotide vaccine of claim 22, wherein at least one codon of the nucleic acid sequence encoding the Amb a1 allergen is modified from a wild type sequence of the Amb a1 allergen to an analogous human codon.

26. A polynucleotide vaccine composition comprising:
a polynucleotide comprising a nucleic acid sequence encoding an antigen derived from a non-host species of a first phylum or first kingdom, wherein the nucleic acid sequence encoding the antigen is modified by deletion of a native signal sequence; and
an immunomodulatory nucleic acid molecule comprising the sequence 5′-cytosine-guanine-3′.

27. The polynucleotide vaccine composition of claim 26, wherein the nucleic acid sequence encoding the antigen is further modified to include a heterologous signal sequence derived from a second phylum or second kingdom, wherein the signal sequence is operably linked to the antigen-encoding sequence.

28. The polynucleotide vaccine composition of claim 27, wherein the heterologous signal sequence comprises a hemagglutinin A (HA) signal sequence.

29. The polynucleotide vaccine composition of claim 26, wherein at least one codon of the nucleic acid sequence encoding the antigen is modified from a wild type sequence of the non-host species to an analogous codon of a host species.

30. The polynucleotide vaccine composition of claim 26, wherein the antigen is Amb a1.

31. The polynucleotide vaccine composition of claim 26, wherein the immunomodulatory nucleic acid molecule comprises a sequence selected from the group consisting of 5′-rrcgyy-3′, 5′-rycgyy-3′, 5′-rrcgyycg-3′, 5′-rycgyycg-3′ or 5′-(TCG)n-3′.

32. The polynucleotide vaccine composition of claim 26, wherein the immunomodulatory nucleic acid molecule comprises a sequence selected from the group consisting of: AACGTT, AGCGTT, GACGTT, GGCGTT, AACGTC, AGCGTC, GACGTC, GGCGTC, AACGCC, AGCGCC, GACGCC, GGCGCC, AACGCT, AGCGCT, GACGCT, GGCGCT, ATCGTT, ACCGTT, GTCGTT, GCCGTT, ATCGTC, ACCGTC, GTCGTC, GCCGTC, ATCGCT, ACCGCT, GTCGCT, GCCGCT, ATCGCC, ACCGCC, GTCGCC, GCCGCC, AACGTTCG, AGCGTTCG, GACGTTCG, GGCGTTCG, AACGTCCG, AGCGTCCG, GACGTCCG, GGCGTCCG, AACGCCCG, AGCGCCCG, GACGCCCG, GGCGCCCG, AACGCTCG, AGCGCTCG, GACGCTCG, GGCGCTCG, ATCGTTCG, ACCGTTCG, GTCGTTCG, GCCGTTCG, ATCGTCCG, ACCGTCCG, GTCGTCCG, GCCGTCCG, ATCGCTCG, ACCGCTCG, GTCGCTCG, GCCGCTCG, ATCGCCCG, ACCGCCCG, GTCGCCCG and GCCGCCCG.
